(19) United States
(12) Patent Application Publication
(10) Pub. No.: US 2010/0055114 A1
(43) Pub. Date: Mar. 4, 2010

(54) USE OF INTEGRIN ALPHA 10 BINDING ANTIBODY TO MODULATE EXTRACELLULAR MATRIX (CARTILAGE) TURNOVER

(76) Inventor: Evy Lundgren-Akerlund, Bjarred (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/448,199

(22) PCT Filed: Dec. 17, 2007
(86) PCT No.: PCT/GB2007/004847

§ 371 Date: Oct. 29, 2009

Related U.S. Application Data
(60) Provisional application No. 60/874,974, filed on Dec. 15, 2006.

(51) Int. Cl. A61K 39/395; C07K 16/18; C07K 2/00; C07K 14/78; A61K 38/02; A61K 38/39; G01N 33/53; A61P 19/10; A61P 19/02

(52) U.S. Cl. 424/175.1; 530/389.1; 530/300; 530/356; 514/2; 514/12; 435/7.21

(57) ABSTRACT

The present invention relates to a novel use of alpha10, or a heterodimer thereof, for affecting cartilage extracellular matrix (ECM) turnover. Further, it relates to the use of a binding entity binding specifically to alpha10, or a heterodimer thereof, in the preparation of a medicament for treating a condition affecting ECM, such as rheumatoid arthritis and osteoarthritis. In particular, the invention relates to a method of treating an individual with a condition affecting ECM turnover, comprising administering to the individual an effective amount of a binding agent entity binding specifically to alpha10, or a heterodimer thereof.

### FIELD OF THE INVENTION

The present invention relates to the use the integrin alpha 10, or a heterodimer comprising the same (for example, a α10β1 heterodimer) for modulating extracellular matrix (ECM) turnover, particularly proteoglycan turnover. Specifically, the invention provides uses of binding entities to alpha 10 for preventing progression of ECM degradation, and in the treatment of conditions involving increased extracellular matrix turnover, such as arthritic diseases.

### BACKGROUND OF THE INVENTION

### Integrins Regulate Diverse Events by Cell-Cell and Cell-Matrix Interactions

Integrins were originally identified as intermediary cell surface structures that linked the internal cytoskeleton with the immediate environment or extracellular cell matrix, and were considered functionally “dead” molecules. This reasoning was partially based on the observation that most integrins contain only a very small cytoplasmic tail lacking any signalling motifs. Today, integrins are known as highly complex structures that via interaction with other cell surface receptors and recruitment of intracellular adapter proteins participate in cell signaling from the inside and out (Phillips et al., 1988, Blood 71:831-43.), from outside and in (Law et al., 1999, Nature 401:808-811.), and have been shown to transduce signals laterally across the cell membrane (Hynes, 2002, Cell 110:673-87; for review see Miranti & Brugge, 2002, Nat Cell Biol 4:E83-90.).

Blocking of some of the best studied integrins using monoclonal antibodies or small molecule inhibitors has been shown to abrogate cell-cell and cell-matrix contacts resulting in intervention of diverse biological processes including development, tissue repair, angiogenesis, inflammation and haemostasis. Some of these antibodies are the subject of clinical phase trials (Shimaoka & Springer, 2003, Nat Rev Drug Discov 2:703-16).

Integrin-mediated immunomodulatory and extracellular matrix modulatory effects have thus far largely been restricted to stimulation of integrins via their native ECM ligands or anti-o integrin antibodies (Loeser, 2002, Biorheology 39:119-24). Such studies have, in analogy with other integrin receptors, demonstrated inside-out, outside-in, and lateral signalling participation of chondrocyte integrins.

### Rheumatoid Arthritis

Rheumatoid arthritis (RA) is an inflammatory, autoimmune disease that affects synovial joints, causing pain, swelling, and reduced mobility for the patient All peripheral joints can be affected in RA, but the most commonly affected are those of the hands, feet and knees. In RA, the immune system attacks the synovium (tissue lining the joint capsule) for unknown reasons, causing local inflammation. The inflammation ultimately results in destruction of cartilage and bone within the joint, as well as the destruction of ligaments, tendons, and muscles that support the joint. In the rheumatoid synovium, activated T cells, B cells, macrophages, fibroblasts, endothelial cells and plasma cells can be identified. Many of the treatments in the pipeline focus on the more targeted inhibition of these specific cells. Although the actual cause of RA is unknown, there is a strong inheritance component.

RA occurs in 0.5-2.0% of the adult population worldwide with one in three patients becoming severely disabled within 20 years. The prevalence of RA is three times greater in women than in men, with a peak age of onset usually between 20 to 45 years. Mortality rates are 27% higher in RA sufferers than in age- and gender-matched controls and even higher in the subset of women. This translates to a reduced life expectancy of somewhere between 5 to 18 years depending on the study. Radiographically evident joint disease is seen in >67% of patients with the first 2 years and >72% of patients within the first 5 years.

Rheumatoid arthritis is initially characterised by an inflammatory response of the synovial membrane conveyed by an influx of a number of different cell types. The lining becomes hyperplastic and expands. In addition, bone destruction is seen.

Disease-modifying drugs are available in RA, but they are of limited use due to side-effects, and many patients do not respond to existing therapy. During the past 20 years a more aggressive approach to the treatment of RA has led to the development of disease-modifying antirheumatic drugs (DMARDS) that attempt to halt disease progression. The most prescribed DMARD is methotrexate, originally used in the treatment of cancer. Others include gold salts, antimalarials, sulphasalazine, tetracyclines and cyclosporine. Physicians usually prescribe DMARDs at disease onset while moderate-to-severe RA suffers are given NSAIDs, corticosteroids and DMARDs concurrently.

### Osteoarthritis

Osteoarthritis (OA) is a progressive, degenerative joint disease and is the most common form of arthritis. It strongly associates with aging and is a major cause of pain and disability in the elderly. A variety of mechanical, metabolic or constitutional insults may trigger OA. Often the insults remain unclear (‘primry’ OA) but sometimes a clear cause such as trauma may be apparent (‘secondary’ OA). All the joint tissues (cartilage, bone, synovium, capsule, ligament, muscle) depend on each other for health and function. Insult to one impacts on the others resulting in a common OA phenotype affecting the whole joint. The OA process involves new tissue production, most notably bone (‘osteophyte’), and remodelling of joint shape. Often OA compensates for the insults, resulting in an anatomically altered but pain-free functioning joint (‘compensated’ OA). Sometimes, however, it fails, resulting in progressive damage, associated symptoms and presentation as an OA patient with ‘joint failure’. Such a perspective explains the clinical heterogeneity of OA and the variable clinical outcomes.

Osteoarthrtis is the leading cause of physical disability over the age of 65 years affecting an estimated 10% of the population. The prevalence of OA-related physical disability is greater in women than men and rises steadily to 25% in women over the age of 85 years. Predictions suggest that there will be a 66% increase in the number of people with OA-related disability by the year 2020. A similar increase in the number of people with severe symptomatic OA of the hip and knee requiring joint replacement surgery in the next 30 years is predicted if disease-modifying strategies for the medical treatment and prevention of OA cannot be found.

Osteoarthritis (OA) is not a single disease or process but rather the clinical and pathological outcome of a range of processes and disorders that lead to structural, and eventually symptomatic, failure of one or more synovial joints. OA-involves the entire joint including the subchondral bone, ligaments, capsule, synovial membrane and periarticular muscles as well as the articular cartilage. Ultimately the articular cartilage degenerates with fibrillation, fissures, ulceration and full thickness loss of the joint surface.

Treatment of osteoarthritis includes a wide spectrum of approaches where most treatments are palliative with the exception of surgery. This means that most treatments relieve pain and thereby increase joint function of the patient, but the treatments do not change the course of the disease. Surgical interventions include joint replacement and osteotomy, which may reverse the progress of osteoarthritis and provide long-term improved function and pain relief for a specific joint.

There is thus an urgent need to treat diseases affecting ECM turnover, such as RA, OA. Existing drug therapies for OA reduce the symptoms (mainly pain), and are only moderately effective and often leave patients with a substantial pain burden. Thus, to date, no drugs are available with proven disease-modifying efficacy in OA.

Accordingly, the present invention seeks to provide novel treatments for conditions affecting ECM-turnover, such as RA and OA.

### SUMMARY OF THE INVENTION

In view of the foregoing disadvantages known in the art when treating a mammal, such as a human being, for a condition affecting ECM turnover, such as e.g. RA or OA, the present invention provides uses of alpha 10, or a heterodimer thereof such as alpha10beta1 (α10β1), to modulate, e.g. to inhibit, prevent or slow down, or reduce degradation and/or increase de novo synthesis, of cartilage ECM, including any molecule of said cartilage ECM, such as e.g. proteoglycan.

A first aspect of the present invention provides the use of alpha10 (α10), or a heterodimer thereof such as alpha10beta1, for affecting cartilage ECM turnover substantially as described herein with reference to the description.

A second aspect of the present invention provides the use of a binding entity binding specifically to alpha 10 or a heterodimer thereof such as alpha10beta1 in the preparation of a medicament for treating a condition where cartilage ECM turnover is affected substantially as described herein with reference to the description.

A third aspect of the present invention provides the use of a binding entity binding specifically to alpha10, or a heterodimer thereof such as alpha10beta1, in the preparation of a diagnostic or prognostic agent for a condition affecting cartilage ECM turnover substantially as described herein with reference to the description.

A fourth aspect of the present invention provides a method for affecting ECM turnover, substantially as described herein with reference to the description.

A fifth aspect of the present invention provides a method for treating an individual with a condition affecting ECM turnover, the method comprising administering to the individual an effective amount of a binding entity substantially as described herein with reference to the description.

A sixth aspect of the present invention provides a method for diagnosing or prognosing a condition affecting ECM turnover in an individual, substantially as described herein with reference to the description.

A seventh aspect of the present invention provides a method for monitoring the progression of a condition affecting ECM turnover substantially as described herein with reference to the description.

### SHORT DESCRIPTION OF THE DRAWINGS

FIG. 1 shows similar antibody response after immunization of both Itga10 KO and WT littermate with type II collagen. Mice were immunized with type II rat collagen in CFA to induce collagen induced arthritis (CIA). Serum was collected before immunization (D0) and 14 days later (D14). Serum levels of anti-collagen II antibodies were measured by immunoassay in duplicate. Shortly, sera were diluted 1:100 in PBS and distributed in a 96 well plate that had been coated with 10 μg/ml (50 μl/well) of rat collagen type II and blocked for unspecific binding with 1% bovine serum albumin. After incubation of the samples overnight at 4° C., alkalin phosphatase-labelled goat anti-mouse antibodies specific for the IgG Fc part was added for detection. Each sample was evaluated in duplicates and the results were expressed in absorbance mean value±s.d.

FIG. 2 shows that Arthritis score is not significantly different for Collagen induced arthritis in alpha10 KO mice. (2a) Arthritis scoring and (2b) incidence are shown. Integrin alpha10 KO mice (KO), heterozygous (Het) mice as well as wild type B10Q littermates (WT) were immunized intradermally at day 0 (D0) with 100 ml type II rat collagen in CFA at a final concentration of 1 mg/ml. At day 35 (D35), indicated by the dotted vertical line, the mice were boosted by intradermal injection of type II rat collagen in IFA.

FIG. 3 shows that Collagen induced arthritis is more cartilage destructive in Itga10 deficient mice. (a) Signs of clinical inflammation during the disease course are represented as mean percent of max histological score. At the endpoint of collagen-induced arthritis (CIA) Itga10 KO (n7-22) and B10Q littermates (n=18) mice were also scored histologically for cartilage destruction, proteoglycan depletion, and osteophyte formation. (b) COMP serum levels were measured at day 0 (before collagen immunization), at day 14 and at day 65 (experiment end-point). Sera were collected and submitted to a 1:100 dilution. COMP levels were quantified by immunoassay in duplicate. Results were expressed in ng/ml (±s.d) calculated on a standard curve using purified COMP. Significance, measured by Mann-Whitney, was reach only at day 65 * P<0.05 (P=0.049). P values at day 0 and at day 14 were respectively of 0.9 and 0.07.

FIG. 4 shows that the inflammatory part of antigen induced arthritis is not different in Itga10KO mice. Immune response towards mBSA was induced by intradermal injection of mBSA/CFA (100 ml at 1 mg/ml) 14 days before intra-articular challenge (represented by the discontinuous vertical line) using 50 mg/knee of mBSA at day 0 (D0). Arthritis in the AIA model was measured as swelling of knee joints. Increase in knee joint size due to inflammation was expressed as the size difference between right knee (injected with mBSA) and left knee (control injected with saline). Itga10 deficient, heterozygous or wild type littermate mice were scored until day 14.

FIG. 5 shows Itga10 deficient mice show a stronger PG depletion in the ALA model than their wt littermates. The severity of arthritis in the knee joints was scored histologically on tissues collected at endpoint of the experiment. Itga10-deficient (KO; n=8) mice were compared with wild type littermate control (WT; n=8). Results are expressed as the mean percent of max histological score±s.d. Both cartilage destruction and osteophyte formation were non-detectable (ND). Significant increase of PG depletion was detected in KO compared with WT control mice ** (P=0.00004).

FIG. 6 shows Four antibody cocktail collagen antibody induced arthritis same in Itga10 KO and wt littermates. Arthritis scoring of all the mice (a) and incidence of all the mice (b) were represented here. Integrin alpha10 subunit KO mice (KO) as well as wild type B10Q littermate (WT) were immunized intravenously at day 0 (D0) with four collagen specific monoclonal antibodies (M2139, CIIC1, UL-1 and CIIC2) recognizing J1, C1, U1 and D3 epitopes on type II collagen. Equal concentrations of each monoclonal antibody were mixed in PBS to achieve a final concentration of 4 mg/mouse. On day 7, LPS at a concentration of 25 ug/mouse, was injected intra-peritoneally. Mouse were observed and scored for arthritis development from day 0 to day 37.

FIG. 7 shows histology of normal and arthritis joint sections from the 4Ab-CAIA experiment staining. Safranin O staining of normal WT ankle (a), clinically inflamed WT ankle (b), normal KO ankle (c) and inflamed KO ankle (d) of mice sacrificed on day 37 after antibody transfer (30 days after LPS injection). Results shown are representative of those obtained from fifteen mice in each group. KO mice had a disease incidence of 100% and the normal KO represented in this staining is from non immunized mice (Original magnifications, ×20).

FIG. 8 shows that Itga10 KO mice show stronger cartilage damage in the CAIA model. Hind paws of 4Ab-CAIA treated mice were histologically scored at day 37. (a) The histological score graph shows the four criteria selected (inflammation, cartilage destruction, PG depletion and osteophyte formation) in order to assess arthritis seventy. The results are shown as mean percentage of max histological score. Osteophyte formation was non-detectable (ND). (b) COMP serum levels were measured at day 0 (before transfer), at day 14 and at day 37 (experiment end-point). Sera were collected and used at a dilution of 1:100. COMP levels were quantified by immunoassay in duplicates. Results were expressed as means of concentrations in ng/ml±s.d. A significant difference in COMP serum levels were measured in KO mice compared to WT littermates at day 14 (P=0.048) and at day 37 (P=0.025) after transfer. At day 0 the difference between KO and WT was not significant (P=0.4). * P<0.05.

FIG. 9 shows that Itga10 KO mice produce less de novo PG after inflammatory cartilage attack. Itga10 KO mice (Right column E-H, KO) as well as wild type B10Q littermate (Left column A-D, WT) were immunized intravenously at Day 0 (D0) with the collagen specific monoclonal antibody UL-1 recognizing the U1 epitope on type II collagen. The monoclonal antibody was diluted in PBS to achieve a final concentration of 4.0 mg/mouse. The mice received a single injection and three randomly picked mice were, sacrificed at day 3, 7 and 21 after antibody transfer. Control mice were sacrificed at day 0 and were not subjected to antibody transfer. Ankles from hind paws were collected, decalcified and paraffin embedded. Sections were stained with toluidine blue (Original magnification ×20).

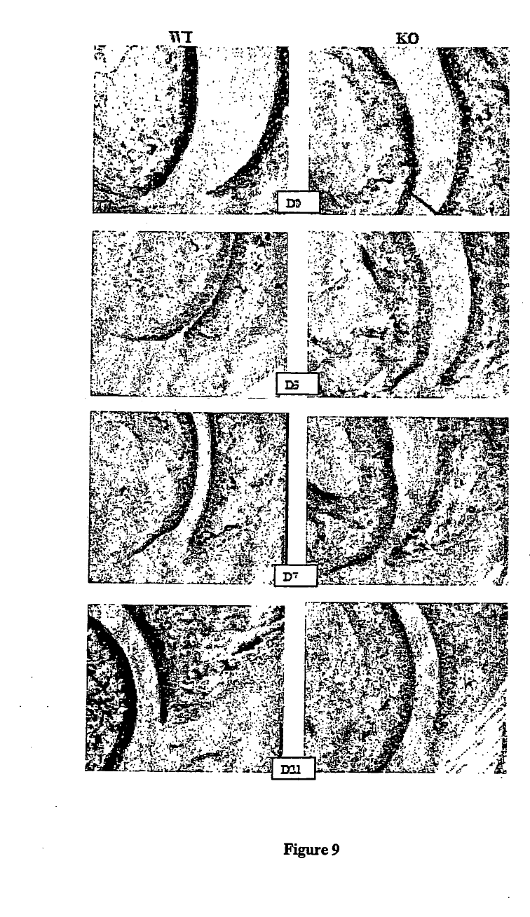

FIG. 10 shows results from exp016. PG release, measured as newly synthesized PG release in media is shown. G06 and E07: P<0.05, A05 P=0.0538, all compared to control antibody CT17.

FIG. 11 shows results from exp014. PG release, measured as newly synthesized PG release in media is shown. A05 P<0.05.

FIG. 12 shows synthesis of proteoglycan in explants as a response to alpha10 IgG4 treatment. Proteoglycan synthesis (4 h 35-S incorporation in cartilage) was analysed after treatment with IgG4 (10 μg/ml) for 48 h. BMP2 (400 ng/ml) was used as a positive controls and IL-1β (10 ng/ml) was used as a control for down-regulation of proteoglycan synthesis. Data from three independent explant experiments using cartilage from one patient with moderate OA (76 y.o.). Mean values with s.e.m are plotted. Statistical analyses: t-test p<0.05 for A05.

### DETAILED DESCRIPTION OF THE INVENTION

### Alpha 10

A newly discovered collagen-binding integrin, alpha10beta1, includes the integrin subunit alpha10 (Camper et al., (1998) J. Biol. Chem. 273:20383-20389). The integrin is expressed on chondrocytes. Expression is initiated at the start of chondrogenesis and continues to be expressed during cartilage development and homeostasis in adults. It associates with the beta1 subunit and forms a receptor capable of binding the cartilage specific type II collagen. Itga10 is expressed in high levels on chondrocytes, which are resident in the articular cartilage, allowing the cells to interact with the surrounding matrix.

Cloning and cDNA sequencing showed that it shares the general structure of other integrin alpha subunits. The predicted amino acid sequence consists of a 1167-amino acid mature protein (see SEQ ID NO:8 below), including a signal peptide (22 amino acids) with sequence amino acid no 1-22 with the sequence MELPFVTHLFLPLVFLTGLCSP [SEQ ID NO:1], a long extracellular domain (1098 amino acids) from amino acid no 23-1120 with the sequence FNLDEHHPRLFPGPPEAEFGYSVLQHVGGGQRWMLVGAPWDGPS GDRRGDVYRCPVGGAHNAPCAKGHLGDYQLGNSSHPAVNMHL GMSLLETDGDGGFMACAPLWSRACGSSVFSSGICARVDASFQPQ GSLAPTAQRCPTYMDVVIVLDGSNSIYPWSEVQTFLRRLVGKLFI DPEQIQVGLVQYGESPVHEWSLGDFRTKEEVVRAAKNLSRREGR ETKTAQAIMVACTEGFSQSHGGRPEAARLLVVVTDGESHDGEELP AALKACEAGRVTRYGIAVLGHYLRRQRDPSSFLREIRTIASDPDER FFFNVTDEAALTDIVDALGDRIFGLEGSHAENESSFGLEMSQIGFS THRLKDGILFGMVGAYDWGGSVLWLEGGHRLFPPRMALEDEFPP ALQNHAAYLGYSVSSMLLRGGRRLFLSGAPRFRHRGKVIAFQLK KDGAVRVAQSLQGEQIGSYFGSELCPLDTDRDGTTDVLLVAAPM FLGPQNKETGRVYVYLVGQQSLLTLQGTLQPEPPQDARFGFAMG ALPDLNQDGFADVAVGAPLEDGHQGALYLYHGTQSGVRPHPAQ RIAAASMPHALSYFGRSVDGRLDLDGDDLVDVAVGAQGAAILLS SRPIVHLTPSLEVTPQAISVVQRDCRRRGQEAVCLTAALCFQVTSR TPGRWDHQFYMRFTASLDEWTAGARAAFDGSGQRLSPRRLRLSV GNVTCEQLHFHVLDTSDYLRPVALTVTFALDNTTKPGPVLNEGSP TSIQKLVPFSKDCGPDNECVTDLVLQVNMDIRGSRKAPFVVRGGR RKVLVSTTLENRKENAYNTSLSLIFSRNLHLASLTPQRESPIKVEC AAPSAHARLCSVGHPVFQTGAKVTFLLEFEFSCSSLLSQVFVKLTA SSDSLERNGTLQDNTAQTSAYIQYEPHLLFSSESTLHRYEVHPYGT LPVGPGPEFKTTLRVQNLGCYVVSGLIISALLPAVAHGGNYFLSLS QVITNNASCIVQNLTEPPGPPVHPEELQHTNRLNGSNTQCQVVRC HLGQLAKGTEVSVGLLRLVHNEFFRRAKFKSLTVVSTFELGTEEG SVLQLTEASRWSESLLEVVQTRPIL [SEQ ID NO:2]; a transmembrane domain (24 amino acids) from amino acid no 1121-1144 with the amino acid sequence ISLWILIGSVLGGLLLLALLVFCLW [SEQ ID NO:3], and a short cytoplasmic domain (22 amino acids) from amino acid no 1145-1167 with the sequence KLGFFAHKKIPEEEKREEKLEQ [SEQ ED NO:4]. In contrast to most alpha-integrin subunits, the cytoplasmic domain of alpha10 does not contain the conserved sequence KXGFF(R/K)R [SEQ ID NO:5]. Instead, the predicted amino acid sequence in alpha10 is KLGFFAH [SEQ ID NO:6]. It is suggested that the GFFKR [SEQ ID NO:7] motif in alpha-chains are important for association of integrin subunits and for transport of the integrin to the plasma membrane (De Melker et al. (1997) Biochem. J. 328529-537). The extracellular part contains a 7-fold repeated sequence, an I-domain (199 amino acids) and three putative divalent cation binding site. Sequence analysis has revealed that the alpha10 subunit is most closely related to the I domain-containing a subunits with the highest identity to alpha 1 (37%), alpha 2 (35%) and alpha 11 (42%).

A first aspect of the present invention provides the use of alpha10, or a heterodimer thereof, for affecting cartilage ECM turnover substantially as described herein with reference to the description.

By “affecting cartilage ECM turnover” we mean to include being able to modulate ECM-turnover by e.g. to inhibit, prevent or slow down, or reduce degradation and/or to increase de novo synthesis, of ECM, or any molecule of ECM, such as e.g. proteoglycan. Examples of molecules of ECM are given in the text herein. By “ECM turnover” we mean to include either de novo ECM synthesis or ECM degradation, or both taken together.

Thus, the invention provides use of integrin subunit alpha10, or a heterodimer thereof, to modulate cartilage extra cellular matrix (ECM) turnover.

Further embodiments are wherein alpha10, or the heterodimer thereof, is used as a target molecule for modulating cartilage ECM turnover.

Still further embodiments are wherein the target molecule is targeted by disruption or wherein the target molecule is targeted by a binding entity.

Even further embodiments are wherein the binding entity is an antibody, or a peptide, or a collagen moiety.

Even further embodiments are wherein the cartilage ECM turnover is modulated to inhibit, prevent or slow down, or reduce degradation of the cartilage ECM.

Still further embodiments are wherein the cartilage ECM turnover is modulated to increase de novo synthesis of the cartilage ECM.

Still further embodiments are wherein any molecule of said cartilage ECM is selected from the group consisting of collagens, proteoglycans and non-protein components.

Even further embodiments are wherein the integrin subunit alpha 10 or a heterodimer thereof is expressed on a cell surface.

To address role of integrin alpha10beta1 in cartilage during an inflammatory insult the inventors evaluated challenged integrin alpha 10 knockout mice (Itga10 KO) using different experimental arthritis models. Arthritis was initiated with either immunization with type II collagen (CII) (collagen induced arthritis, CIA), methylated BSA (antigen induced arthritis, AIA), or with the transfer of monoclonal antibodies specific for CII (collagen antibody induced arthritis, CAIA). No difference in the macroscopic severity of arthritis was seen. Histological analysis showed no difference in the inflammatory response of the formation of a pannus tissue. However, the Itga10 KO mice showed a more pronounced tissues destruction, measured as depletion of proteoglycans (PG) and cartilage degradation in all three arthritis models studied. There was however no difference in osteophyte formation in integrin alpha10 defective mice compared to WT mice. It was concluded that the absence of the Itga10 does not affect the onset or the course of induced arthritis in any of the animal models studied here. In addition, histological scoring for inflammation in AIA, CIA and CAIA experiments showed that the number of over whole joint invading cells in the interstitial joint space were similar in the WT and Itga10 KO mice. However, enhanced reduction in PG content in Itga10 KO mice compared to WT mice was conclusive from all the performed RA experiments indicating the importance of integrin alpha10beta1 for the integrity of the cartilage.

### ECM

Elements constituting the cartilage ECM are synthesis by the chondrocytes. The matrix is organized into an intricate network around and near the cells, and is stabilized by several interactions between molecules within the matrix. The major constituents of the cartilage matrix are collagens, proteoglycans and non-proteins components.

The collagens present in articular cartilage are collagen type II, VI, IX, X and XI. The most abundant is type II collagen representing about 90% of the collagens. This fibril-forming collagen is secreted and associate with type IX collagen and type XI collagen to form fibres.

Aggrecan is the shortened name of the large aggregating chondroitin sulphate proteoglycan (PG). Aggrecan, which is one of the most widely studied proteoglycans, is abundant; it represents up to 10% of the dry weight of cartilage (articular cartilage is up to 75% water). It binds to hyaluronic acid and link-protein to form large molecular weight aggregates. This negatively charged molecule is responsible for water and electrolytes retention in the ECM conferring to the articular cartilage its compression-tolerance properties.

The cartilage ECM is also constituted of relatively large but less abundant non-collagenous protein components including cartilage oligomeric matrix protein (COMP), thrombospondin (TSP). All these elements of the ECM are further interacting with each other via the presence of smaller member of the ECM proteins such as fibromodulin, decorin, biglycan, chondroadherin and fibulin.

The ECM proteins combine with each other and form a relatively dense network. Their disposition and their interaction lead to a cartilaginous tissue highly resistant to mechanical stress. Collagens are described as responsible for cartilage resistance and proteoglycans are accounted for cartilage resiliency.

When the integrity of the articular cartilage is altered, as it happens in arthritis, small fragments of ECM molecules are released and can be found circulating in the blood/serum. Several of these fragments are today used as biomarkers to evaluate if cartilage degradation is taking place. One of the cartilage protein fragments measured in the serum is COMP, and its increased concentrations has been correlated to an active, ongoing joint disease.

Several of the cartilage matrix components will bind directly to chondrocytes mainly via integrins. The α₁β₁, α₂β₁, α₁₀β₁ and α₁₁β₁ integrins will interact with type II collagen. The α₁β₁ and α₂β₁ have been described to also mediate adhesion to type VI collagen and matrilin-1. Other chondrocytes cell-surface receptors such as CD44 have been reported to bind specifically to the ECM component hyaluronan.

The chondrocyte matrix is divided in territories according to the proximity to the cell. The matrix immediately surrounding the cells is referred to as the pericellular matrix; next to this is the territorial matrix; the matrix found between the chondrocytes is referred to as the interstitial matrix.

Chondrocytes originate from mesenchymal cell lineage. They constitute the only cells found in the cartilage and represent 5% of the whole cartilage volume. They produce extra-cellular matrix (ECM) proteins and are therefore responsible for primary production of the cartilaginous matrix. They also have the capacity to produce cartilage degrading proteinases allowing them to enzymatically change the composition of the ECM. The equilibrium between synthesis and removal/digestion of ECM components provides stability to cartilage.

It is also important to mention that molecule diffusion through articular cartilage, mechanical stress detection, and integrins interaction with their surrounding are all highly dependent on the ECM composition, density and organization. Studies on diffusion through articular cartilage have shown that PG removal altered the diffusion of non-charged molecules of increasing molecular weight. The diffusion was inversely proportional to PG content.

The ECM may be affected in several ways via alpha10, either on protein level or on gene level. By using a binding entity with binding specificity to alpha10, or a heterodimer thereof, ECM may be affected.

In one embodiment, the binding entity is an antibody, or antigen-binding fragment, or variant, fusion or derivative thereof. By “antibody” we include substantially intact antibody molecules, as well as different types of recombinant antibodies, i.e. chimaeric antibodies, humanised antibodies, human antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same. For example, the antibody may be a monoclonal antibody. The antibody may be generated by traditional hybridoma technology or recombinant technologies as described below.

Thus, in one embodiment the antibody or antigen-binding fragment, or variant, fusion or derivative thereof, comprises or consists of an intact antibody.

For example, the antibody or antigen-binding fragment, or a variant, fusion or derivative thereof, may consist essentially of an intact antibody. By “consist essentially of” we mean that the antibody or antigen-binding fragment, variant, fusion or derivative thereof consists of a portion of an intact antibody sufficient to retain binding specificity for an integrin α10 subunit.

The term ‘antibody’ also includes all classes of antibodies, including IgG, IgA, IgM, IgD and IgE. In one embodiment, however, the antibody is an IgG molecule, such as an IgG1, IgG2, IgG3, or IgG4 molecule.

In a further embodiment, the antibody is a non-naturally occurring antibody. Of course, where the antibody is a naturally occurring antibody, it is provided in an isolated form (i.e. distinct from that in which it is found in nature).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent-parented antibody (Morrison et al (1984) Proc. Natl. Acad. Sci. USA 81, 6851-6855).

Antigenic specificity is conferred by variable domains and is independent of the constant domains, as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

Thus, by “antigen-binding fragment” we mean a functional fragment of an antibody that is capable of binding to an integrin α10 subunit, or a heterodimer thereof.

Exemplary antigen-binding fragments may be selected from the group consisting of Fv fragments (e.g. single chain Fv and disulphide-bonded Fv), Fab-like fragments (e.g. Fab fragments, Fab′ fragments and F(ab)₂ fragments), single antibody chains (e.g. heavy or light chains), single variable domains (e.g. V_{H} and V_{L} domains) and domain antibodies (dAbs, including single and dual formats [i.e. dAb-linker-dAb]).

In one embodiment, the antigen-binding fragment is an scFv.

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

Also included within the scope of the invention are modified versions of antibodies and an antigen-binding fragments thereof e.g. modified by the covalent attachment of polyethylene glycol or other suitable polymer, and uses of the same.

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299, which are incorporated herein by reference) or generation of monoclonal antibody molecules by cell lines in culture. These include, but are not limited to, the hybridoma technique, the human B-cell hybridoma technique, and the Epstein-Barr virus (EBV)-hybridoma technique (see Kohler et al., 1975. Nature 256:4950497; Kozbor et al., 1985. J. Immunol. Methods 81:3142; Cote et al., 1983. Proc. Natl. Acad. Sci. USA 80:2026-2030; Cole et al., 1984. Mol. Cell. Biol. 62:109-120, which are incorporated herein by reference).

The antibody or antigen-binding fragment or derivative thereof may be produced by recombinant means.

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982), which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy or diagnostics, humanised antibodies may be used. Humanised forms of non-human (e.g. murine) antibodies are genetically engineered chimaeric antibodies or antibody fragments having preferably mini-portions derived from non-human antibodies. Humanised antibodies include antibodies in which complementary determining regions of a human antibody (recipient antibody) are replaced by residues from a complementary determining region of a non human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596, which are incorporated herein by reference).

Methods for humanising non-human antibodies are well known in the art Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-1536; U.S. Pat. No. 4,816,567, which are incorporated herein by reference) by substituting human complementarity determining regions with corresponding rodent complementarity determining regions. Accordingly, such humanised antibodies are chimaeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95, Soderlind et al., 2000, Nat Biotechnol 18:852-6 and WO 98/32845 which are incorporated herein by reference).

By “binding specificity” for an integrin α10 subunit, or a heterodimer thereof it is included a binding entity which is capable of binding to an integrin α10 subunit, or a heterodimer thereof such as a α10β1 heterodimer, and not binding to other molecules such as the integrin.

In one embodiment, the integrin α10 subunit, or the heterodimer thereof is localised on the surface of a cell in cartilage, such as articular cartilage. One example of such a cell is a chondrocyte.

In a further embodiment, the binding entity is capable of binding to an integrin α10 subunit, fragments or variants thereof, or a heterodimer thereof in vivo, i.e. under the physiological conditions in which an integrin α10 subunit exists inside the body. Such binding specificity may be determined by methods well known in the art, such as e.g. ELISA, immunohistochemistry, immunoprecipitation, Western blots, chromatography and flow cytometry using transfected cells expressing the α10 subunit or a heterodimer thereof. Examples of how to measure specificity of an antibody is given in e.g. Harlow & Lane, “Antibodies: A Laboratory”, Cold Spring Harbor Laboratory Press, New York, which is incorporated herein by reference.

In a preferred embodiment, the binding entity selectively binds to an integrin alpha-10 subunit Conveniently, the binding moiety selectively binds to a polypeptide comprising or consisting of the amino acid sequence of SEQ ID NO: 8 or natural variants thereof.

SEQ ID NO: 8

MELPFVTHLFLPLVFLTGLCSPFNLDEHHPRLFPGPPEAEFGYSVLQHVG
GGQRWMLVGAPWDGPSGDRRGDVYRCPVGGAHNAPCAKGHLGDYQLGNSS
HPAVNMHLGMSLLETDGDGGFMACAPLWSRACGSSVFSSGICARVDASFQ
PQGSLAPTAQRCPTYMDVVIVLDGSNSIYPWSEVQTFLRRLVGKLFIDPE
QIQVGLVQYGESPVHEWSLGDFRTKEEVVRAAKNLSRREGRETKTAQAIM
VACTEGFSQSHGGRPEAARLLVVVTDGESHDGEELPAALKACEAGRVTRY
GIAVLGHYLRRQRDPSSFLREIRTIASDPDERFFFNVTDEAALTDIVDAL
GDRIFGLEGSHAENESSFGLEMSQIGFSTHRLKDGILFGMVGAYDWGGSV
LWLEGGHRLFPPRMALEDEFPPALQNHAAYLGYSVSSMLLRGGRRLFLSG
APRFRHRGKVIAFQLKKDGAVRVAQSLQGEQIGSYFGSELCPLDTDRDGT
TDVLLVAAPMFLGPQNKETGRVYVYLVGQQSLLTLQGTLQPEPPQDARFG
FAMGALPDLNQDGFADVAVGAPLEDGHQGALYLYHGTQSGVRPHPAQRIA
AASMPHALSYFGRSVDGRLDLDGDDLVDVAVGAQGAAILLSSRPIVHLTP
SLEVTPQAISVVQRDCRRRGQEAVCLTAALCFQVTSRTPGRWDHQFYMRF
TASLDEWTAGARAAFDGSGQRLSPRRLRLSVGNVTCEQLHFHVLDTSDYL
RPVALTVTFALDNTTKPGPVLNEGSPTSIQKLVPFSKDCGPDNECVTDLV
LQVNMDIRGSRKAPFVVRGGRRKVLVSTTLENRKENAYNTSLSLIFSRNL
HLASLTPQRESPIKVECAAPSAHARLCSVGHPVFQTGAKVTFLLEFEFSC
SSLLSQVFVKLTASSDSLERNGTLQDNTAQTSAYIQYEPHLLFSSESTLH
RYEVHPYGTLPVGPGPEFKTTLRVQNLGCYVVSGLIISALLPAVAHGGNY
FLSLSQVITNNASCIVQNLTEPPGPPVHPEELQHTNRLNGSNTQCQVVRC
HLGQLAKGTEVSVGLLRLVHNEFFRRAKFKSLTVVSTFELGTEEGSVLQL
TEASRWSESLLEVVQTRPILISLWILIGSVLGGLLLLALLVFGLWKLGFF
AHKKIPEEEKREEKLEQ
Amino acids 1-1167

The term ‘amino acid’ as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

By “natural variants” we include, for example, allelic variants. Typically, these will vary from the given sequence by only one or two or three, and typically no more than 10 or 20 amino acid residues. Typically, the variants have conservative substitutions.

Also included in “natural variants” is a splice variant of integrin alpha 10, for example as described in WO 99/51639. The splice removed from the extracellular domain is the amino acid sequence VQNLGCYVVSGLIISALLPAVAHGGNYFLSLSQVITNN [SEQ ID NO:9]. The remaining alpha10 sequence is the short version of the alpha10 molecule representing one natural variant of alpha10.

Variants of the above polypeptide sequence include polypeptides comprising a sequence with at least 60% identity to the amino acid of SEQ ID NO: 8, preferably at least 70% or 80% or 85% or 90% identity to said sequences, and more preferably at least 95%, 96%, 97%, 98% or 99% identity to said amino acid sequences.

Percent identity can be determined by methods well known in the art, for example using the LALIGN program (Huang and Miller, Adv. Appl. Math. (1991) 12:337-357) at the Expasy facility site (http://www.ch.embnet.org/software/LALIGN_form.html) using as parameters the global alignment option, scoring matrix BLOSUM62, opening gap penalty −14, extending gap penalty −4.

Alternatively, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may also alternatively be carried out using the Clustal W program (as described in Thompson et al., 1994, Nuc. Acid Res. 22:4673-4680).

The parameters used may be as follows:
Fast pairwise alignment parameters: K-tuple(word) size; 1, window size; 5, gap penalty; 3, number of top diagonals; 5. Scoring method: x percent
Multiple alignment parameters: gap open penalty; 10, gap extension penalty; 0.05.
Scoring matrix: BLOSUM.

Alternatively, the BESTFIT program may be used to determine local sequence alignments.

In one embodiment, polypeptide binding entities, such as antibodies, peptides etc., described herein comprise or consist of L-amino acids.

It will be appreciated by persons skilled in the art that the integrin α10 subunit may be a human or animal integrin α10 subunit In one embodiment, the integrin α10 subunit is human. Examples of known integrin α10 subunits are disclosed in International Patent Application (Publication) No. WO 99/51639 (to Cartela A B), which is incorporated herein by reference.

In another embodiment, the binding entity is capable of binding to an integrin α10 subunit, or a heterodimer thereof selectively. By “capable of binding selectively” we include such antibody-derived binding moieties which bind at least 10-fold more strongly to integrin α10 subunit or a heterodimer thereof than to another proteins (in particular other integrins, such as α11, α1 and α2 having most identity with α10); for example at least 50-fold more strongly or at least 100-fold more strongly. The binding entity may be capable of binding selectively to integrin α10 subunit or a heterodimer thereof under physiological conditions, e.g. in vivo. Suitable methods for measuring relative binding strengths include immunoassays, for example where the binding moiety is an antibody (see Harlow & Lane, “Antibodies: A Laboratory”, Cold Spring Harbor Laboratory Press, New York, which is incorporated herein by reference). Alternatively, binding may be assessed using competitive assays or using Biacore® analysis (Biacore International AB, Sweden).

In a further embodiment, the binding entity binds exclusively to an integrin α10 subunit or a heterodimer thereof.

Exemplary antibodies with binding affinity for the integrin alpha-10 subunit are described in International Patent Application No. PCT/SE2004/000580 (Publication No. WO 2004/089990, incorporated here in by reference). One example of a binding entity therein is the antibody mAb365 binding to the extracellular I-domain of alpha10 (hybridoma deposited at accession number DSM ACC2583 at Deutche Sammlung von Microorganismen und Zellkulturen GmbH).

Another example of binding entities is peptides. Examples of peptides binding to the alpha10 subunit are given in PCT/GB2006/003015 incorporated herein by reference. Examples of peptides are isolated polypeptides capable of binding to an integrin I-domain wherein the polypeptide comprises an amino acid sequence selected from the following group:

| | |
|---|---|
| GIWFENEW; | [SEQ ID NO: 10] |
| WIWPDSGW; | [SEQ ID NO: 11] |
| WENWDGWG; | [SEQ ID NO: 12] |
| and/or | |
| WEDGWLHA. | [SEQ ID NO: 13] |

or a variant, fusion or derivative thereof, or a fusion of a said variant or derivative thereof.

By “antigen-binding fragment” we mean a functional fragment of an antibody that is capable of binding to the integrin alpha-10 subunit or a heterodimer thereof.

Preferably, the antigen-binding fragment is selected from the group consisting of Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains), domain antibodies (dAbs, including single and dual formats [i.e. dAb-linker-dAb]) and nanobodies (for example, see Revets et al., 2005, Expert Opin Biol Ther. 5(1):111-24).

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

It will be appreciated by persons skilled in the art that invention encompasses variants, fusions and derivatives of binding entities, such as antibodies and peptides, and fusions of said variants or derivatives, as well as uses thereof provided that such variants, fusions and derivatives retain binding specificity for an integrin α10 subunit or heterodimer thereof.

Variants may be made using the methods of protein engineering and site-directed mutagenesis well known in the art using the recombinant polynucleotides (see example, see Molecular Cloning: a Laboratory Manual, 3rd edition, Sambrook & Russell, 2001, Cold Spring Harbor Laboratory Press, which is incorporated herein by reference).

By ‘fusion’ of said polypeptide we include a polypeptide fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any variant or derivative of said polypeptide are also included in the scope of the invention. It will be appreciated that fusions (or variants or derivatives thereof) which retain desirable properties, such as retain binding specificity for an integrin α10 subunit or heterodimer thereof, are preferred.

By “epitope” it is herein intended to mean a site of a molecule to which an antibody binds, i.e. a molecular region of an antigen. An epitope may be a linear epitope, which is determined by e.g. the amino acid sequence, i.e. the primary structure, or a three-dimensional epitope, defined by the secondary structure, e.g. folding of a peptide chain into beta sheet or alpha helical, or by the tertiary structure, e.g. the way which helices or sheets are folded or arranged to give a three-dimensional structure, of an antigen.

The fusion may comprise a further portion which confers a desirable feature on the said polypeptide of the invention; for example, the portion may be useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

By ‘variants’ of the polypeptide alpha10, or SEQ No 8, or polypeptide binding entities such as antibodies, or any other polypeptide described herein, we include insertions, deletions and substitutions, either conservative or non-conservative. In particular we include variants of the polypeptide where such changes do not substantially alter the activity of the said polypeptide. For example, we include variants of the polypeptide where such changes do not substantially alter the binding specificity for an integrin α10 subunit or heterodimer thereof.

The polypeptide variant may have an amino acid sequence which has at least 75% identity with one or more of the amino acid sequences given above, for example at least 80%, at least 90%, at least 95%, at least 96%, at least 97%, at least 98%, at least 99%, or 100% identity with one or more of the amino acid sequences specified above.

Once suitable binding entities are defined, e.g. antibodies or peptides, they may be tested for activity, such as binding specificity or a biological activity of the antibody, for example by ELISA, immunohistochemistry, flow cytometry, immunoprecipitation, western blots, etc. The biological activity may be tested in different assays with readouts for that particular feature. Examples of one or more biological activity of the binding entities, e.g. antibodies, according to the invention are ability to modulate ECM, for example protection of cartilage from degradation e.g. protection of reduction in proteoglycan content.

Examples of suitable assays for testing said biological activity is known in the art and also given in the examples herein, such as e.g. Saffranin-O staining of tissue to evaluate proteoglycan content.

The polypeptide, variant, fusion or derivative of the invention may comprise one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which are capable of binding an integrin α10 subunit. The term ‘peptidomimetic’ refers to a compound that mimics the conformation and desirable features of a particular peptide as a therapeutic agent.

For example, the polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond.

It will be appreciated that the polypeptide may conveniently be blocked at its N- or C-terminus so as to help reduce susceptibility to exoproteolytic digestion.

A variety of uncoded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, exemplary polypeptides comprise tonal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminus cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. For example, the peptides of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods are discussed and disclosed in U.S. Pat. No. 6,008,058, which is incorporated herein by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), which is incorporated herein by reference, has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present Polypeptide cyclisation is also a useful modification and is preferred because of the stable structures formed by cyclisation and in view of the biological activities observed for cyclic peptides.

Thus, in one embodiment the polypeptide binding moiety is cyclic. However, in an alternative embodiment, the polypeptide is linear.

In another embodiment, the invention provides for a use of an agent or binding entity capable of modulating, such as increasing or decreasing the expression of an integrin alpha-10 subunit in the preparation of an agent for modulating ECM. The agent is administered to a subject in need thereof in an amount sufficient to affect any condition affecting ECM integrity, such as e.g. proteoglycan content.

Thus, in one aspect of the invention, the present invention can be used to inhabit, prevent, reduce or slow down the progression of ECM degradation. Another aspect of the invention is to increase de novo synthesis of ECM, such as de novo synthesis of proteoglycan.

By “expression” we mean the production of integrin alpha-10 polypeptide subunits, i.e. alpha-10 protein production. It will be appreciated by persons skilled in the art that modulation of expression may be accomplished by a modulatory effect at a number of different stages of the gene expression pathway, for example inhibition or induction of the synthesis of mRNA (‘transcription’) or translation of mRNA into polypeptide sequences (‘translation’).

By “inhibition” or “increasing” or “induction” we mean that expression of integrin alpha-10 subunit gene (mRNA and/or protein) is inhibited, increased, or induced to express in whole or in part. For example, a compound, such as a binding entity, may inhibit, increase, or induce expression of the integrin alpha-10 subunit by at least 10%, preferably at least 20%, 30%, 40%, 50%, 60%, 70%, 80% or 90%, and most preferably by 100% compared to expression of the integrin alpha-10 subunit before modulation of the cells which have not been exposed to a compound or binding entity. In a one embodiment, the compound or binding entity is capable of inhibiting, increasing, or inducing expression of the integrin alpha-10 subunit by 50% or more compared to expression of the integrin alpha-10 subunit in cells which have not been exposed to the compound or binding entity.

Advantageously, the binding entity is capable of inhibiting, increasing, or inducing the expression of the integrin alpha-10 subunit under physiological conditions, for example in vivo (i.e. in the body of a patient suffering from a malignant tumour).

Assays for testing whether a compound is capable of inhibiting, increasing, or inducing integrin alpha-10 subunit expression are well known in the art. For example, a tissue sample (slice) may be taken and in situ hybridisation studies performed to determine the level of expression of integrin alpha-10 subunit mRNA (see Sambrook & Russell, 2000, Molecular Cloning, A Laboratory Manual, Third Edition, Cold Spring Harbor, N.Y.). Alternatively, or in addition, integrin alpha-10 protein levels may be measured using anti-alpha-10 antibodies, for example by western blotting.

In one embodiment, the binding entity is capable of inhibiting, increasing, or inducing the expression of an integrin alpha-10 subunit selectively.

By ‘selectively’ we mean that the binding entity inhibits, increases, or induces expression of the integrin alpha-10 subunit to a greater extent than it modulates the expression of other molecules in the ECM. Preferably, the compound inhibits, increases, or induces only the expression of the integrin alpha-10 subunit, although it will be appreciated that the expression and activity of other proteins within the cells may change as a downstream consequence of a selective inhibition of the integrin alpha-10 expression. Thus, we exclude agents which have a non-specific effect on gene expression in a cell.

Advantageously, the compound is also selective in the sense that it acts preferentially on expression of the integrin alpha-10 subunit in a cell in or nearby cartilage, such as a chondrocyte (i.e. cell-specific inhibition). In one embodiment, the binding entity inhibits, increases, or induces expression of the integrin alpha-10 subunit in chondrocytes only.

In a one embodiment of the above aspects of the invention, the binding entity is capable of inhibiting, increasing, or inducing the transcription of an integrin alpha-10 subunit. By “transcription” we mean the process whereby the DNA sequence in a gene is copied into mRNA.

In an alternative preferred embodiment of the above aspects of the invention, the binding entity is capable of inhibiting, increasing, or inducing the translation of an integrin alpha-10 subunit. By “translation” we mean the process that occurs at the ribosome whereby the information in mRNA is used to specify the sequence of amino acids in a polypeptide chain.

Thus, the invention provides the use of a binding entity as defined herein to inhibit, increase, or induce the expression of an integrin alpha-10 subunit in a cell, such as a chondrocyte cell.

In a particularly preferred embodiment, the binding entity comprises or consists of a nucleic acid molecule.

In one embodiment, the nucleic acid is SEQ ID 14 (see below).

In a particularly preferred embodiment, the nucleic acid molecule comprises or consists of the nucleotide sequence of SEQ ID NO:14 or a fragment thereof or the complementary sequence thereto, or a variant of the same.

```
SEQ ID NO: 14
      1 gggaaagtga agaaaacaga aaaggagagg gacagaggcc agaggacttc tcatactgga
    61 cagaaaccga tcaggcatgg aactcccctt cgtcactcac ctgttcttgc ccctggtgtt
  121 cctgacaggt ctctgctccc cctttaacct ggatgaacat cacccacgcc tattcccagg
  181 gccaccagaa gctgaatttg gatacagtgt cttacaacat gttgggggtg gacagcgatg
  241 gatgctggtg ggcgccccct gggatgggcc ttcaggcgac cggagggggg acgtttatcg
  301 ctgccctgta gggggggccc acaatgcccc atgtgccaag ggccacttag gtgactacca
  361 actgggaaat tcatctcatc ctgctgtgaa tatgcacctg gggatgtctc tgttagagac
  421 agatggtgat gggggattca tggcctgtgc ccctctctgg tctcgtgctt gtggcagctc
  481 tgtcttcagt tctgggatat gtgcccgtgt ggatgcttca ttccagcctc agggaagcct
  541 ggcacccact gcccaacgct gcccaacata catggatgtt gtcattgtct tggatggctc
  601 caacagcatc tacccctggt ctgaagttca gaccttccta cgaagactgg tagggaaact
  661 gtttattgac ccagaacaga tacaggtggg actggtacag tatggggaga gccctgtaca
  721 tgagtggtcc ctgggagatt tccgaacgaa ggaagaagtg gtgagagcag caaagaacct
  781 cagtcggcgg gagggacgag aaacaaagac tgcccaagca ataatggtgg cctgcacaga
  841 agggttcagt cagtcccatg ggggccgacc cgaggctgcc aggctactgg tggttgtcac
  901 tgatggagag tcccatgatg gagaggagct tcctgcagca ctaaaggcct gtgaggctgg
  961 aagagtgaca cgctatggga ttgcagtcct tggtcactac ctccggcggc agcgagatcc
1021 cagctctttc ctgagagaaa ttagaactat tgccagtgat ccagatgagc gattcttctt
1081 caatgtcaca satgaggctg ctctgactga cattgtggat gcactaggag atcggatttt
1141 tggccttgaa gggtcccatg cagaaaacga aagctccttt gggctggaaa tgtctcagat
1201 tggtttctcc actcatcggc taaaggatgg gattcttttt gggatggtgg gggcctatga
1261 ctggggaggc tctgtgctat ggcttgaagg aggccaccgc cttttccccc cacgaatggc
1321 actggaagac gagttccccc ctgcactgca gaaccatgca gcctacctgg gttactctgt
1381 ttcttccatg cttttgcggg gtggacgccg cctgtttctc tctggggctc ctcgatttag
1441 acatcgagga aaagtcatcg ccttccagct taagaaagat ggggctgtga gggttgccca
1501 gagcctccag ggggagcaga ttggttcata ctttggcagt gagctctgcc cattggatac
1561 agatagggat ggaacaactg atgtcttact tgtggctgcc cccatgttcc tgggacccca
1621 gaacaaggaa acaggacgtg tttatgtgta tctggtaggc cagcagtcct tgctgaccct
1681 ccaaggaaca cttcagccag aaccccccca ggatgctcgg tttggctttg ccatgggagc
1741 tcttcctgat ctgaaccaag atggttttgc tgatgtggct gtgggggcgc ctctggaaga
1801 tgggcaccag ggagcactgt acctgtacca tggaacccag agtggagtca ggccccatcc
1861 tgcccagagg attgctgctg cctccatgcc acatgccctc agctactttg gccgaagtgt
1921 ggatggtcgg ctagatctgg atggagatga tctggtcgat gtggctgtgg gtgcccaggg
1981 ggcagccatc ctgctcagct cccggcccat tgtccatctg accccatcac tggaggtgac
2041 cccacaggcc atcagtgtgg ttcagaggga ctgtaggcgg cgaggccaag aggcagtctg
2101 tctgactgca gccctttgct tccaagtgac ctcccgtact cctggtcgct gggatcacca
2161 attctacatg aggttcaccg catcactgga tgaatggact gctggggcac gtgcagcatt
2221 tgatggctct ggccagaggt tgtcccctcg gaggctccgg ctcagtgtgg ggaatgtcac
2281 ttgtgagcag ctacacttcc atgtgctgga tacatcagat tacctccggc cagtggcctt
2341 gactgtgacc tttgccttgg acaatactac aaagccaggg cctgtgctga atgagggctc
2401 acccacctct atacaaaagc tggtcccctt ctcaaaggat tgtggccctg acaatgaatg
2461 tgtcacagac ctggtgcttc aagtgaatat ggacatcaga ggctccagga aggccccatt
2521 tgtggttcga ggtggccggc ggaaagtgct ggtatctaca actctggaga acagaaagga
2581 aaatgcttac aatacgagcc tgagtctcat cttctctaga aacctccacc tggccagtct
2641 cactcctcag agagagagcc caataaaggt ggaatgtgcc gccccttctg ctcatgcccg
2701 gctctgcagt gtggggcatc ctgtcttcca gactggagcc aaggtgacct ttctgctaga
2761 gtttgagttt agctgctcct ctctcctgag ccaggtcttc gtgaagctga ctgccagcag
2821 tgacagcctg gagagaaatg ggacccttca agataacaca gcccagacct cagcctacat
2881 ccaatatga8 ccccacctcc tgttctctag tgagtctacc ctgcacegct atgaggttca
2941 cccaiatggg accctcccag tgggtcctgg cccagaattc aaaaccactc tcagggttca
3001 gaacctaggc tgctatgtgg tcagtggcct catcatctca gccctccttc cagctgtggc
3061 ccatgggggc aattacttcc tatcactgtc tcaagtcatc actaacaatg caagctgcat
3121 agtgcagaac ctgactgaac ccccaggccc acctgtgcat ccagaggagc ttcaacacac
3181 aaacagactg aatgggagca atactcagtg tcaggtggtg aggtgccacc ttgggcagct
3241 ggcaaagggg actgaggtct ctgttggact attgaggctg gttcacaatg aatttttccg
3301 aagagccaag ttcaagtccc tgacggtggt cagcaccttt gagctgggaa ccgaagaggg
3361 cagtgtccta cagctgactg aagcctcccg ttggagtgag agcctcttgg aggtggttca
3421 gacccggcct atcctcatct ccctgtggat cctcataggc agtgtcctgg gagggttgct
3481 cctgcttgct ctccttgtct tctgcctgtg gaagcttggc ttctttgccc ataagaaaat
3541 ccctgaggaa gaaaaaagag aagagaagtt ggagcaatga atgtagaata agggtctaga
3601 aagtcctccc tggcagcttc ttcaagagac ttgcataaaa gcagaggttt gggggctcag
3661 atgggacaag aagccgcctc tggactatct ccccagacca gcagcctgac ttgacttttg
3721 agtcctaggg atgctgctgg ctagagatga ggctttacct cagacaagaa gagctggcac
3781 caaaactagc catgctccca ccctctgctt ccctcctcct cgtgatcctg gttccatagc
3841 caacactggg gcttttgttt ggggtccttt tatccccagg aatcaataat ttttttgcct
3901 aggtgcctga ctcctttcag attccctctt tatcttccct cacagtttgg aaaggatgag
3961 ggttatcttc ctcgattctt ccaccctctc actttcctgc ctgttcccca ctccacagga
4021 gggagctgac gttggcttga aaggagtaaa gtcaacatct gctgctttcc tgtggactct
4081 ggtgattcat agagccggat ggggagagtc aacaggaaaa aaggagggag gaggaaaagc
4141 cacaagagac attctgtaca attccaagga acagagaagc ctttagacag gcaactgcca
4201 tcccccctga aacctgagac ctgtagtgca ctcgaccgcc ctcaggtgtt ggtgaaacag
4261 agctgccccc aggctcgctg ggcataggct tcctgattcc aagccttttc tgggagcaaa
4321 gccagggcct ggtgcctgat tttctgaagc caggagccct caggtggctg gagctggaat
4381 agcagggagg actgggtgta cctaggcagt attttctcta cttctctcaa gtcttatact
4441 cactcttgag ccctccttgg ggcctgctta gaaagcagac aggagagaga gtactgctac
4501 ttgatgatgg gaaatgcttt cactttacca gctttgggaa gcagcagccc catgggatct
4561 aaaagtgtgg agtctgcatt aagaaaccta catgggtggc atggggctct ggggagcaag
4621 cccttacttg ctcagcactg gttatgtagc acaaatagct cctaggaaaa tgtttctggg
4681 gcaaccctag aaccctggtc atattttgca gggtttctct ggtggaatca gtttgccagc
4741 ccttgcttga tgcttactgg aaatctccag gttaatttct atctctgatc cctceccaac
4801 ccactccata tttgggtcat ggacagtaaa ggcagttgga ttctcataga caactgggta
4861 acttatattt ctttgtaatc aagacttgag atatcgaagt cagttattgg tctccagagt
4921 gcagctctgg gagccttttg aagaatcagc actcattaag agctgagaag agagaagacc
4981 tgattgggtg gttgactagc agtcacagaa cctgtcctcc caggctgttc ctgaggcctg
5041 accacagtat ttattttggc atgtctctgg ccttctgcag aggcccaccc tcatgggcat
5101 tgtctctgtt tcccagtggg gtggacagta tatcagatgg tcagaacaaa t&aagttcag
5161 tgtcaaatga aaaaaaaaaa aaaaaaaaa
```

By “variant” we mean that the nucleotide sequence shares at least 60% identity to the nucleotide sequence of SEQ ID NO: 14 or a fragment thereof, preferably at least 70% or 80% or 85% or 90% identity to said sequence, and more preferably at least 95%, 96%, 97%, 98% or 99% identity to said nucleotide sequence.

Also included are “natural variants”, by which we include, for example, allelic variants. Typically, these will vary from the given sequence by only one or two or three, and typically no more than 10 or 20 amino acid residues. Typically, the variants have conservative substitutions.

Also included in “natural variants” is a splice variant of integrin alpha 10, for example as described in WO 99/51639.

By “nucleic acid molecule” we include DNA, RNA and synthetic oligonucleotides, as well as analogues, conjugates and derivatives thereof. The nucleic acid molecule may be double-stranded or single-stranded.

Other examples of nucleic acid molecules include phosphorothioate oligonucleotides, 2′-O-methoxyethyl phosphorothioate oligonucleotides, 2′-O-methyl oligonucleotides, morpholino oligonucleotides, peptide nucleic acids (‘PNA’) and locked nucleic acid RNA analogues (‘LNA’) (for example, see Da Ros et al., 2005, Curr Med Chem. 12:71-88 and references cited therein).

Nucleic acid molecules for use in the invention may be made by methods well known to persons skilled in the art (for example, see Sambrook & Russell, 2000, Molecular Cloning, A Laboratory Manual, Third Edition, Cold Spring Harbor, N.Y.). For example, the nucleic acid molecules may be synthesised chemically or produced using a cloning vector.

Nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for further manipulation of nucleic acids according to the invention, such as, e.g., subcloning, labelling probes (e.g., random-primer labelling using Klenow polymerase, nick translation, amplification), sequencing, hybridization and the like are well described in the scientific and patent literature, see, e.g., Sambrook & Russell, supra; Current protocols in molecular biology, Ausubel, ed. John Wiley & Sons, Inc., New York (1997); Laboratory techniques in biochemistry and molecular biology: hybridization with nucleic acid probes, Part I. Theory and Nucleic Acid Preparation, Tijssen, ed. Elsevier, N.Y. (1993).

In still a further embodiment, alpha10 expression is restored by gene transfer, such as e.g. in gene therapy. The expression of the exogenous genetic material in vivo, is often referred to as “gene therapy”. Cells to express the alpha10beta1 expression in vivo in cartilage may be e.g. chondrocytes, MSCs, macrophages, monocytes, synovial cells, tenocytes, myoblasts, osteoblasts; and fibroblasts. Disease states and procedures for which such treatments have application include any genetic disorders and diseases where cartilage ECM is affected, such as disorders and diseases of joints, e.g. RA or OA. Cell delivery of the transformed cells may be effected using various methods and includes infusion and direct depot injection into joints, periosteal, bone marrow and subcutaneous sites.

In one embodiment, a composition, such as a therapeutic composition or pharmaceutical composition is administered as an administration vehicle, comprising said monoclonal antibody or a fragment thereof in combination with other gene or bio delivery systems. The combined administration vehicle comprising said monoclonal antibody or a fragment thereof may be used in combination with other gene or bio delivery systems to selectively target cells of interest, e.g. chondrocyte cells. Such a vehicle would involve coupling a binding entity, e.g. an antibody or a fragment thereof to a delivery vehicle which would include, for example, virus, liposomes, microcapsules, nanocapsules, plasters, sublingual tablets, and polymer matrices such as poly(orthoesters), polylactide-polyglycolide polymers, and coupling the treatment agent, e.g. a nucleic acid or alpha10 either to the antibody or a fragment thereof, or to the delivery vehicle.

In one embodiment, the binding entity, such as a peptide or antibody or a fragment thereof will be used as a vehicle to enable targeted gene-delivery of agents to cells of interest in the cartilage, such as e.g. chondrocytes. Cells to be targeted for gene-delivery include cells as mentioned above as well as cells of the skeletal system comprising, cartilage, bone, tendon, ligament and muscle.

In another embodiment a gene is delivered into a cartilage cell, such as a chondrocyte, using a virus, viral vectors including retroviruses, adenoviruses, adeno-associated viruses (AAV), herpes simplex virus and lentivirus.

In one embodiment, the alpha10 gene is transferred using adenovirus and monoclonal antibodies such as the mAb365 antibody. This may be done as described in Barry et al 2003 and Parrott et al 2003, incorporated herein by reference.

In one embodiment the alpha10 gene is delivered into a cell, such as a chondrocyte, by a non-viral method. Non-viral delivery systems include the use of naked DNA, cationic liposomes, cationic lipids and polymers as well as DNA cationic liposome/polycation complexes.

In one embodiment the binding entity. E.g. a peptide or antibody or a fragment thereof may be used in conjunction with a viral or non-viral delivery system for the in vivo transfer of a gene(s) directly to the damaged tissue, e.g. of cartilage, tendon, bone, ligament, muscle etc. The binding entity and the gene(s) of interest may be delivered locally to the site of tissue damage.

In brief, expression vectors may be constructed comprising a nucleic acid molecule which is capable, in an appropriate host cell, of expressing the polypeptide binding moiety or compound encoded by the nucleic acid molecule.

A variety of methods have been developed to operably link nucleic acid molecules, especially DNA, to vectors, for example, via complementary cohesive termini. For instance, complementary homopolymer tracts can be added to the DNA segment to be inserted into the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, e.g. generated by endonuclease restriction digestion, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerising activities.

A desirable way to modify the DNA encoding the polypeptide of alpha10, or the heterodimer, is to use PCR. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The combination of these activities therefore generates blunt-ended DNA segments. The blunt-ended segments are then incubated with a larger molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc., New Haven, Conn., USA.

A further embodiment of the first aspect of the invention provides the use of a binding entity comprising a target cell specific portion with binding affinity for a cell of interest, such as a chondrocyte.

By “target cell specific” portion we mean a portion of the compound which comprises one or more binding sites which recognise and bind to entities on the target cell. Upon contact with the target cell, the target cell specific portion may be internalised along with the portion capable modulating alpha-10 expression.

The entities recognised by the target cell-specific portion are expressed predominantly, and preferably exclusively, on the target cartilage cell. The target cell specific portion may contain one or more binding sites for different entities expressed on the sane target cell type, or one or more binding sites for different entities expressed on two or more different target cell types.

Preferably, the target cell-specific portion recognises the target cell with high avidity.

By “high avidity” we mean that the target cell-specific portion recognises the target cell with a binding constant of at least K_{d}=10⁻⁶ M, preferably at least K_{d}=10⁻⁹M, suitably K_{d}=10⁻¹⁰ M, more suitably K_{d}=10⁻¹¹ M, yet more suitably still K_{d}=10⁻¹² M, and more preferably K_{d}=10⁻¹⁵ M or even K_{d}=10⁻¹⁸ M.

The entity which is recognised may be any suitable entity which is expressed by cartilage cells. Often, the entity which is recognised will be an antigen.

A second aspect of the present invention provides the use of a binding entity binding specifically to alpha 10 or a heterodimer thereof in the preparation of a medicament for treating a condition where cartilage ECM turnover is affected substantially as described herein with reference to the description.

Still a further aspect provides a binding entity binding specifically to integrin subunit alpha 10 or a heterodimer thereof for treating a condition where cartilage ECM turnover is affected.

One further aspect of the present invention provides a pharmaceutical composition comprising the binding entity of claim 10 for treating a condition where cartilage ECM turnover is affected. The present invention also includes compositions comprising pharmaceutically acceptable acid or base addition salts of a binding entity of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3-naphthoate)] salts, among others.

Pharmaceutically acceptable base addition salts may also be used to produce pharmaceutically acceptable salt forms of the compounds according to the present invention.

The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The binding entities described herein may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

It will be further appreciated by persons skilled in the art that binding entities, e.g. the antibodies and antigen-binding fragments, variants, fusions and derivatives thereof, described herein may exist in monomeric form or in the form of a homo- or hetero-multimer thereof (e.g. dimer, trimer, tetramer, pentamer, etc.).

A third aspect of the present invention provides the use of a binding entity binding specifically to alpha10, or a heterodimer thereof in the preparation of a diagnostic or prognostic agent for a condition affecting cartilage ECM turnover substantially as described herein with reference to the description. Thus, one aspect provides the use of a binding entity binding specifically to alpha10, or a heterodimer thereof; in the preparation of a diagnostic or prognostic agent for a condition affecting cartilage ECM turnover.

Further embodiments of the methods, uses, compositions of the invention include wherein the condition where cartilage ECM turnover is affected is OA or RA.

Still further embodiments are wherein the binding entity is an antibody.

In a further embodiment of the invention, the binding entity comprises a therapeutic and/or detectable moiety.

By a “detectable moiety” we include the meaning that the moiety is one which, when located at the target site following administration of the compound of the invention into a patient, may be detected, typically non-invasively from outside the body and the site of the target located. The detectable moiety may be a single atom or molecule which is either directly or indirectly involved in the production of a detectable species. Thus, the binding agents of this embodiment of the invention are useful in imaging and diagnosis.

Suitable detectable moieties are well known in medicinal chemistry and the linking of these moieties to polypeptides and proteins is well known in the art. Examples of detectable moieties include, but are not limited to, the following: radioisotopes (e.g. ³H, ¹⁴C, ³⁵S, ¹²³I, ¹²⁵I, ¹³¹I, ⁹⁹Tc, ¹¹¹In, ⁹⁰Y, ¹⁸⁸Re), radionuclides (e.g. ¹¹C, ¹⁸F, ⁶⁴Cu), fluorescent labels (e.g. FITC, rhodamine, lanthanide phosphors, carbocyanine), enzymatic labels (e.g. horseradish peroxidase, β-galactosidase, luciferase, alkaline phosphatase), chemiluminescent, biotinyl groups and predetermined polypeptide epitopes recognised by a secondary reporter (e.g. leucine zipper pair sequences, binding sites for secondary antibodies, metal binding domains, epitope tags). In some embodiments, labels are attached by spacer arms of various lengths to reduce potential steric hindrance.

The radio- or other labels may be incorporated in the polypeptides of the invention in known ways. For example, if the binding moiety is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57, which is incorporated herein by reference) can be used to incorporate ¹²³I. Reference (“Monoclonal Antibodies in Immunoscintigraphy”, J-F Chatal, CRC Press, 1989, which is incorporated herein by reference) describes other methods in detail.

The binding entity as described above have efficacy in the treatment of a condition where cartilage ECM is affected, such as e.g. RA, OA and the like.

By ‘treatment’ we include both therapeutic and prophylactic treatment of a subject/patient. The term ‘prophylactic’ is used to encompass the use of a polypeptide or formulation described herein which either prevents or reduces the likelihood of a condition affecting ECM in a patient or subject.

For example, the binding entity may have efficacy in the treatment of a condition selected from the group consisting of arthritic diseases (such as rheumatoid arthritis and osteoarthritis), joint inflammation, inflammation-induced cartilage destruction, multiple sclerosis, ankylosing spondylitis, psoriatic arthritis and autoimmune chronic inflammatory diseases.

In one embodiment, the binding entity has efficacy in the treatment an arthritic disease, such as rheumatoid arthritis or osteoarthritis. Such efficacy may be determined in suitable animal models, such as arthritis models in mice (see Examples).

For example, the binding entity may be capable of modulating (e.g. inhibiting) the degradation ECM, such as of collagen, in vitro and/or in vivo. Example is inhibition, slowing down, reducing of proteoglycan degradation.

Thus, the invention provides the following methods:
a method for affecting ECM turnover, substantially as described herein with reference to the description.
a method for treating an individual with a condition affecting ECM turnover, the method comprising administering to the individual an effective amount of a binding entity substantially as described herein with reference to the description.
a method for diagnosing or prognosing a condition affecting ECM turnover in an individual, substantially as described herein with reference to the description
a method for monitoring the progression of a condition affecting ECM turnover substantially as described herein with reference to the description.

In particular, said method for diagnosing or prognosing a condition affecting ECM turnover in an individual, the method comprises administering to the individual an effective amount of a binding entity, as described herein.

A further aspect of the present invention describes a method for imaging ECM bearing cartilage cells expressing an integrin α10 subunit or heterodimer thereof associated with an condition affecting ECM, in the body of an individual, the method comprising administering to the individual an effective amount of a binding entity as defined herein.

In one embodiment of the above aspects of the invention, the method further comprises the step of detecting the location of the binding entity in the individual.

A seventh aspect of the invention provides a method for monitoring the progression of a condition affecting ECM in an individual, the method comprising:
(a) providing a sample of cells collected from the individual at a first time point and measuring the amount of integrin α10 subunit protein therein using a binding entity as described herein;
(b) providing a sample of cells collected from the individual at a second time point and measuring the amount of integrin α10 subunit protein therein using a binding entity as described herein; and
(c) comparing the amount of integrin α10 subunit protein measured in steps (a) and (b)
   wherein an decreased amount of integrin α10 subunit protein measured in step (b) compared to step (a) is indicative of a progression in the inflammatory condition.

An eighth aspect of the invention provides a method for identifying cells associated with a condition affecting ECM, the method comprising measuring the amount of integrin α10 subunit protein in a sample of cells to be tested using a binding entity as described herein and comparing it to the amount of integrin α10 subunit protein to a positive and/or negative control. The positive control may comprise cells from a subject who is suffering from a condition affecting ECM and the negative control may comprise cells from a healthy subject who is not suffering from said condition.

In one embodiment of the invention, the cells are selected from the group consisting of cartilage cells, such as chondrocyte cells.

The amount of integrin α10 subunit in a sample may be determined using methods well known in the art Suitable methods for assaying integrin α10 protein levels in a biological sample include e.g. antibody-based techniques. For example, integrin α10 protein expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g. with urea and neutral detergent, for the liberation of integrin α10 protein for western blot or dot/slot assay (Jalkanen et al., 1985, J. Cell. Biol. 101:976-985; Jalkanen et al., 1987, J. Cell. Biol. 105:3087-3096, which are incorporated herein by reference). In this technique, which is based on the use of cationic solid phases, quantitation of integrin α10 protein can be accomplished using isolated integrin α10 protein as a standard. This technique can also be applied to body fluids.

In one embodiment, the cells to be tested are identified as cells associated with a condition affecting ECM by the downregulation of integrin α10 subunit protein levels compared to corresponding normal healthy cells. By “downregulated” we mean that the integrin α10 subunit protein is decreased by at least 10% compared to expression of the integrin in normal (healthy) cells. For example, the level of the integrin α10 subunit protein may be decreased by at least 20%, 30%, 40%, 50%, or even 100% or more.

In a further embodiment, the above methods further comprise the step of detecting the location of the binding entity in the individual.

Detecting the binding entity can be achieved using methods well known in the art of clinical imaging and diagnostics. The specific method required will depend on the type of detectable label attached to the binding entity. For example, radioactive atoms may be detected using autoradiography or in some cases by magnetic resonance imaging (MRI) as described above.

In a further embodiment of the above methods and uses of the invention, the condition affecting cartilage ECM is selected from the group consisting of arthritic diseases (such as rheumatoid arthritis and osteoarthritis), joint inflammation, and inflammation-induced cartilage destruction. For example, the condition affecting ECM may be an arthritic disease, such as rheumatoid arthritis or osteoarthritis.

Persons skilled in the art will further appreciate that the medicaments and agents described above have utility in both the medical and veterinary fields. Thus, the medicaments and agents may be used in the treatment of both human and non-human animals (such as horses, dogs, mice, rats, apes, monkeys, pigs, and cats). Preferably, however, the patient is human.

Also described herein is a pharmaceutical composition comprising a binding entity as described herein, or comprising a nucleic acid as described herein and a pharmaceutically acceptable excipient, diluent or carrier.

As used herein, ‘pharmaceutical composition’ means a therapeutically effective formulation.

A ‘therapeutically effective amount’, or ‘effective amount’, or ‘therapeutically effective’, as used herein, refers to that amount which provides a therapeutic effect for a given condition and administration regimen (for example, an amount sufficient to inhibit the degradation of collagen). This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

It will be appreciated by persons skilled in the art that such an effective amount of the compound or formulation thereof may be delivered as a single bolus dose (i.e. acute administration) or, more preferably, as a series of doses over time (i.e. chronic administration).

It will be further appreciated by persons skilled in the art that the binding entity or the nucleic acid for use in medicine according to the invention may be administered in combination with one or more other conventional agents for the treatment of inflammatory conditions.

For example, in the case of rheumatoid arthritis, suitable conventional agents include, but are not limited to, disease-modifying antirheumatic drugs (DMARDS, e.g. methotrexate), gold salts, antimalarials, sulphasalazine, tetracyclines, cyclosporine, NSAIDs, corticosteroids, Leflunomide (Arava; Aventis), tumour-necrosis factor-α (INFα) inhibitors, such as etanercept (Enbrel; Amgen), infliximab (Remicade; J&J/Centocor) and adalimumab (Humira; Abbott).

In the case of OA, suitable conventional agents include, but are not limited to analgesics or anti-inflammatory agents, such as acetaminophen (also known as paracetamol), or non-steroidal anti-inflammatory drugs (NSAIDs) inhibiting cyklooxygenase (COX), such as COX2 inhibitors, Disease-Modifying Osteoarthritis Drugs (DMOADs), such as MMP inhibitors, or interleukin 1 (IL-1) inhibitor.

The binding entity, according to one aspect of the invention may be formulated at various concentrations, depending on the efficacy/toxicity of the compound being used. In one embodiment, the formulation comprises the agent of the invention at a concentration of between 0.1 μM and 1 mM, for example between 1 μM and 100 μM, between 5 μM and 50 μM, between 10 μM and 50 μM, between 20 μM and 40 μM or about 30 μM. For in vitro applications, formulations may comprise a lower concentration of a compound of the invention, for example between 0.0025 μM and 1 μM.

Thus, there is provided a pharmaceutical formulation comprising an amount of a binding agent effective to treat a condition affecting ECM (as described above).

It will be appreciated by persons skilled in the art that the medicaments and agents will generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA, which is incorporated herein by reference).

For example, the medicaments and agents can be administered orally, buccally or sublingually in the form of tablets, capsules, ovules, elixirs, solutions or suspensions, which may contain flavouring or colouring agents, for immediate-, delayed- or controlled-release applications. The medicaments and agents may also be administered via intracavernosal injection.

For gene therapy methods of administering the nucleic acid to the patient is known in the art.

Such tablets may contain excipients such as microcrystalline cellulose, lactose, sodium citrate, calcium carbonate, dibasic calcium phosphate and glycine, disintegrants such as starch (for example, corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Exemplary excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol propylene glycol and glycerin, and combinations thereof.

The medicaments, agents, compositions and binding entities can also be administered parenterally, for example, intravenously, intra-articularly, intra-arterially, intraperitoneally, intra-thecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (for example, to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the medicaments and agents will usually be from 1 to 1000 mg per adult (i.e. from about 0.015 to 15 mg/kg), administered in single or divided doses.

It is appreciated that for the prevention or treatment of disease, the appropriate dosage of a binding entity, such as an antibody or peptide or even nucleic acid will depend on the type of disease to be treated, the severity and of course of the disease, whether the antibody or a fragment thereof is administered for preventative or therapeutic purposes, the course of previous therapy and the patient's clinical history and response to the antibody or a fragment thereof. The binding entity according to the present invention is suitably administered to the patient at one time or over a series of treatments. Depending on the type and severity of the disease, about 0.015 to 15 mg of antibody or a fragment thereof/kg of patient weight is an initial candidate dosage for administration to the patient. Administration may be, for example, by one or more separate administrations, or by continuous infusion. For repeated administrations over several days or longer, depending on the condition, the treatment is repeated until a desired suppression or alleviation of the disease symptoms occurs. However, other dosage regimens may be useful and are not excluded.

The effectiveness of a binding entity such as peptide, an antibody, antigen binding fragment, variant, fusion or derivative thereof in alleviating the symptoms, preventing or treating disease may be improved by serial administering or administration in combination with another agent that is effective for the same clinical indication, such as another peptide, antibody or a fragment thereof directed against a different epitope than that of the antibody according to the invention, or one or more conventional therapeutic agents known for the intended therapeutic indication.

Suitable pharmaceutically acceptable agents affecting such indications may be ant-inflammatory drugs such as non steroidal anti-inflammatory drugs (NSAIDS) for the treatment of diseases where ECM is affected such as arthritic diseases e.g. osteoarthritis, rheumatoid arthritis; anti-cytoltine agents e.g. anti-TNF antibodies, interleukin receptor antagonist, matrix metalloproteinase (MMP) inhibitors or bone morphogenic proteins (BMP); local anaesthetics for use post-operatively following orthopaedic surgery for the treatment of pain management or hypolipidemic drugs for treatment of atherosclerotic plaque, matrix metalloproteinase (MMPs) inhibitors or bone morphogenic proteins (BMPs).

The medicaments and agents can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations may be arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the medicaments and agents can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route.

For application topically to the skin, the medicaments and agents can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Where the medicament or agent is a polypeptide, it may be preferable to use a sustained-release drug delivery system, such as a microsphere. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Sustained-release immunoglobulin compositions also include liposomally entrapped immunoglobulin Liposomes containing the immunoglobulin are prepared by methods known per se. See, for example Epstein et al., Proc. Natl. Acad. Sci. USA 82: 3688-92 (1985); Hwang et al., Proc. Natl. Acad. Sci. USA 77: 4030-4 (1980); U.S. Pat. Nos. 4,485,045; 4,544,545; 6,139,869; and 6,027,726, which are incorporated herein by reference. Ordinarily, the liposomes are of the small (about 200 to about 800 Angstroms), unilamellar type in which the lipid content is greater than about 30 mole percent (mol. %) cholesterol; the selected proportion being adjusted for the optimal immunoglobulin therapy.

Alternatively, polypeptide medicaments and agents can be administered by a surgically implanted device that releases the drug directly to the required site.

Electroporation therapy (EPT) systems can also be employed for the administration of proteins, polypeptides and nucleic acids. A device which delivers a pulsed electric field to cells increases the permeability of the cell membranes to the drug, resulting in a significant enhancement of intracellular drug delivery.

Proteins, polypeptides and nucleic acids can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and polypeptide delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Pharmaceuticals as described herein can also be delivered orally. One such system employs a natural process for oral uptake of vitamin B12 in the body to co-deliver proteins and polypeptides. By riding the vitamin B12 uptake system, the protein or polypeptide can move through the intestinal wall. Complexes are produced between vitamin B12 analogues and the drug that retain both significant specificity for intrinsic factor (IF) in the vitamin B12 portion of the complex and significant bioactivity of the drug portion of the complex.

Further aspects of the present invention provide:
A method for affecting cartilage ECM turnover, the method comprising the steps of
   a) providing cartilage ECM comprising cartilage cells
   b) targeting integrin subunit alpha10, or a heterodimer thereof, during sufficient time to affect the cartilage ECM turnover.
methods according to above, wherein the cartilage ECM is provided as a tissue sample, or a cell suspension, or as an individual such as a human being or any mammal.
methods according to above, wherein the integrin subunit alpha10, or a heterodimer thereof, is targeted by a binding entity or by disruption.
methods according to above, wherein the binding entity is an antibody, or a peptide, or a collagen moiety.
Methods according to above, the methods further comprising c) detecting cartilage ECM turnover after and optionally before step b) above targeting the integrin subunit alpha10, or a heterodimer thereof.
A method for treating an individual wherein said individual has a condition affecting cartilage ECM turnover, the method comprising the steps of
   (a) providing an individual, said individual having a condition affecting cartilage turnover,
   (b) administering to the individual an effective amount of a binding entity targeting the integrin subunit alpha10, or a heterodimer thereof.
A method for detecting a condition affecting cartilage ECM turnover in an individual, the method comprising the steps of
   a) providing cartilage ECM comprising cartilage cells,
   b) targeting integrin subunit alpha10 or a heterodimer thereof with a binding entity,
   c) detecting said binding entity,
   d) scoring cartilage ECM turnover compared to a reference sample,
   thereby detecting said condition affecting cartilage ECM turnover.
A method for diagnosing or prognosing a condition affecting ECM turnover in an individual, the method comprising the steps of
   (a) providing cartilage ECM comprising cartilage cells
   (b) targeting integrin subunit alpha10 or a heterodimer thereof with a binding entity,
   (c) detecting said binding entity,
   (d) scoring cartilage ECM turnover compared to a reference sample,
   (e) relating said scoring in d) to a diagnosis or a prognosis of a condition affecting ECM turnover.
The methods according to above, wherein the targeting is in vitro, in situ or in vivo.
The methods according to above, wherein the targeting is in vivo and the detection are performed by in vivo imaging.
A method for monitoring the progression of a condition affecting cartilage ECM turnover in an individual, the method comprising:
   (a) providing a sample of cartilage cells collected from the individual at a first time point,
   (b) targeting said cells collected from the individual at a first time point for integrin alpha10 subunit, or heterodimer thereof, with a binding entity,
   (c) detecting said binding entity on cells collected from the individual at a first time point,
   (d) measuring the amount of integrin alpha10 subunit, or heterodimer thereof, on cells collected from the individual at a first time point,
   (e) providing a sample of cells collected from the individual at a second point,
   (f) targeting cells collected from the individual at a second time point for integrin α10 subunit, or heterodimer thereof, with a binding entity,
   (g) detecting said binding entity on cells collected from the individual at a second time point,
   (h) measuring the amount of integrin alpha10 subunit, or heterodimer thereof, on cells collected from the individual at a second time point,
   (i) comparing the amount of integrin alpha10 subunit, or heterodimer thereof, measured in steps d) and h) above, thereby monitoring the progression of a condition affecting ECM turnover in an individual.
A method for detecting cartilage cells associated with a condition affecting ECM, the method comprising the steps of
   (a) providing a sample of cartilage cells from or nearby cartilage ECM,
   (b) targeting integrin alpha10 subunit or a heterodimer thereof collected from an individual with a binding entity,
   (c) detecting said binding entity in b) above, thereby detecting cells associated with a condition affecting ECM.
methods according to above, wherein the condition where cartilage ECM turnover is affected is OA or RA.
Use of alpha10, or a heterodimer thereof, for affecting cartilage ECM turnover substantially as described herein with reference to the description.
Use of a binding entity binding specifically to alpha 10 or a heterodimer thereof in the preparation of a medicament for treating a condition where cartilage ECM turnover is affected substantially as described herein with reference to the description
Use of a binding entity binding specifically to alpha10, or a heteromer thereof, in the preparation of a diagnostic or prognostic agent for a condition affecting cartilage ECM turnover substantially as described herein with reference to the description.
A method for affecting ECM turnover, substantially as described herein with reference to the description.
A method for treating an individual with a condition affecting ECM turnover, the method comprising administering to the individual an effective amount of a binding entity substantially as described herein with reference to the description.
A method for detecting or diagnosing or prognosing a condition affecting ECM turnover in an individual, substantially as described herein with reference to the description.
A method for monitoring the progression of a condition affecting ECM turnover substantially as described herein with reference to the description.

The binding entities for use in the methods of the invention may be provided in the form of a kit comprising a pharmaceutical composition as described above. Thus, a kit may be provided for use in the treatment of a condition where ECM is affected.

Alternatively, the kit may comprise a detectable binding entity as described herein suitable for use in diagnosis. Such a diagnostic kit may comprise, in an amount sufficient for at least one assay, the diagnostic agent as a separately packaged reagent Instructions for use of the packaged reagent are also typically included. Such instructions typically include a tangible expression describing reagent concentrations and/or at least one assay method parameter such as the relative amounts of reagent and sample to be mixed, maintenance time periods for reagent/sample admixtures, temperature, buffer conditions and the like.

As used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

Preferred, non-limiting examples which embody certain aspects of the invention will now be described.

### EXAMPLES

In the present examples, alpha 10 integrin KO mice backcrossed on an arthritis-susceptible mouse background. These mice were used in several different experimentally induced arthritis models. Arthritis was induced in different manner in order to have a large variety of approaches to rheumatoid arthritis and a better understanding of the role of alpha 10 integrin in disease onset and development.

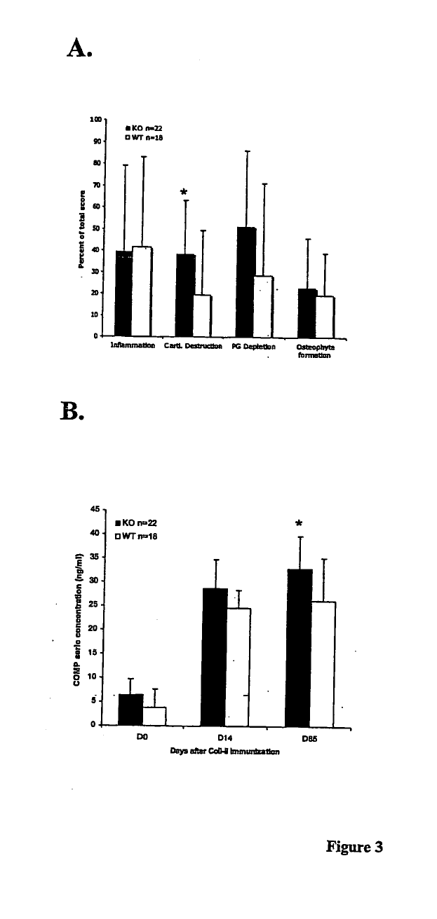

Arthritis was first induced by injection of type II collagen as antigen. This model is very efficient in inducing disease and can lead to a chronic arthritis. In this case the disease course was followed for 65 consecutive days. In this model it was seen a tendency for higher PG depletion and significant higher level of cartilage destruction in alpha 10 KO mice compared to WT mice (FIG. 3a). These observations were confirmed by measurement of COMP in the serum, showing that COMP levels were significantly higher in KO mice than in WT mice at the experiment endpoint (day 65) (FIG. 3b).

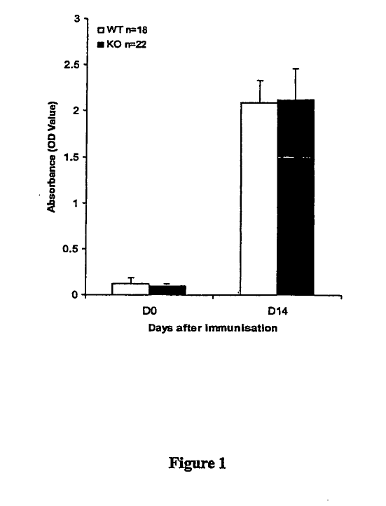

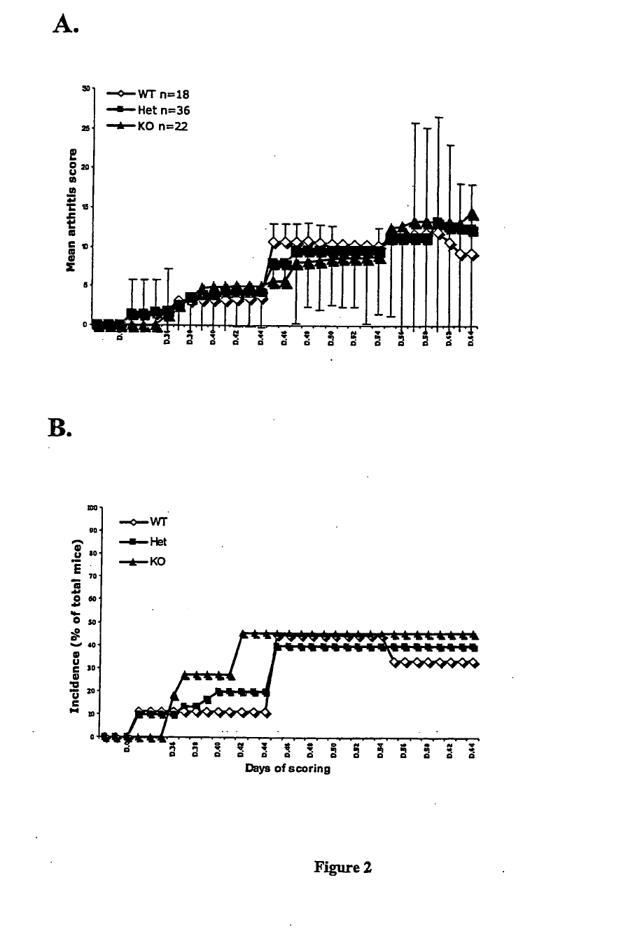

The alpha 10 integrin did not appear to be involved in the inflammatory processes, since the absence of alpha 10 integrin did not seem to influence swelling and redness of the paws, both hallmarks of arthritis (FIG. 3a). Both the onset and chronicity of the disease were similar for KO and WT mice (FIG. 2a) and the antibody response to type II collagen was not compromised in alpha 10 integrin deficient mice (FIG. 1).

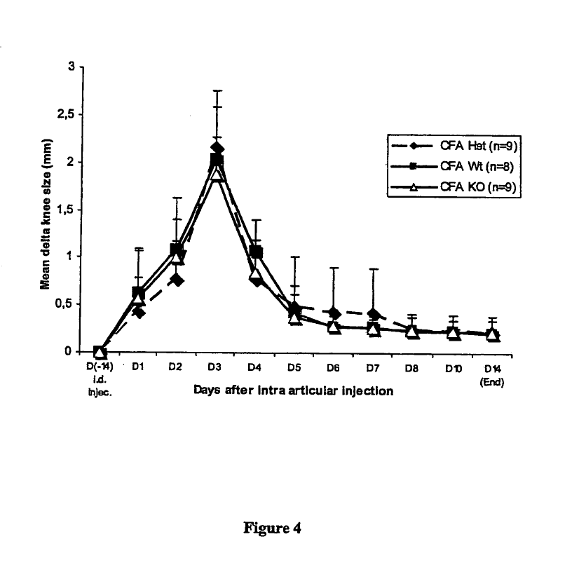

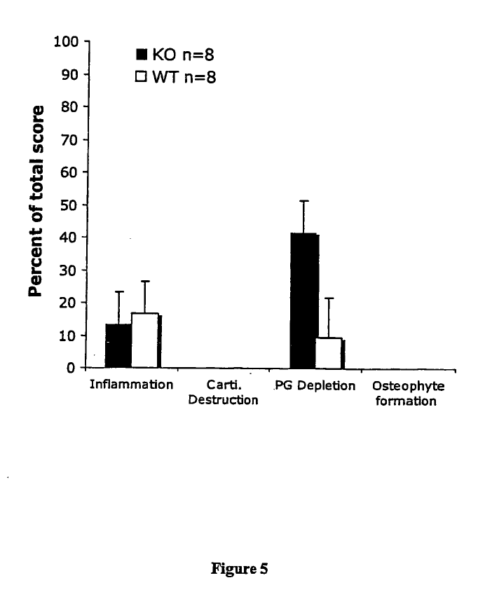

In a further attempt to understand the role of the alpha 10 integrin in arthritis models, mice were injected directly in the knees with mBSA. This antigen-induced arthritis (AIA) model is a well characterized experimental model with an acute onset phase one day after antigen second injection starting by joint swelling and infiltration of different cells of the immune system followed by a chronic phase with pannus formation and cartilage degradation. As in the CIA experiment, it was also found an increased PG depletion in KO compared with WT littermate (FIG. 5). Also in this model, the absence of alpha 10 integrin did not affect the development of mBSA-induced arthritis (FIG. 4b). The AIA model has been described to rely on Th1 cells during the acute phase as essential pathogenic component during acute AIA (Pohlers, 2004). Hence the fact that the invasion of cells into the joint space was not affected by the absence of a10b1 showed that this integrin have no obvious effect on cell migration and homing into the joint.

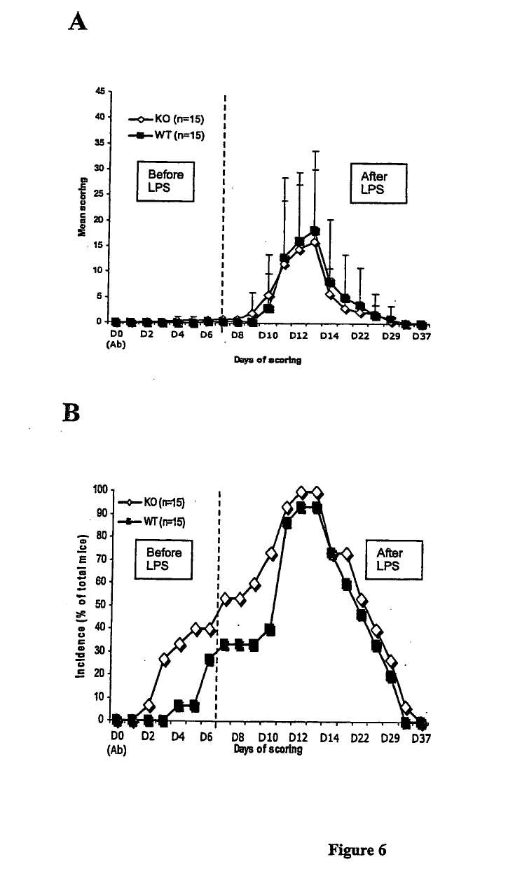

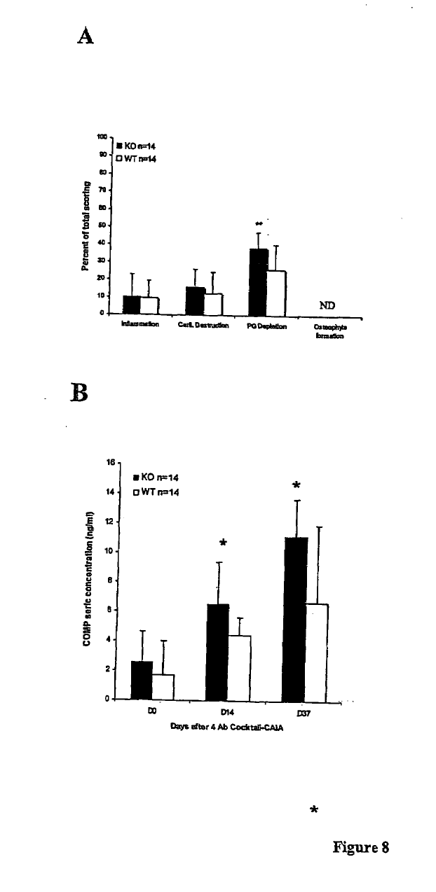

The third model collagen antibody induced arthritis, is a model where no immunization is made and the mice are injected directly with specific antibodies mimicking the effector phase of the disease. Antibodies directed against type II collagen were transferred by intravenous injection into the tail vein. Lipopolysaccharides (LPS) was injected at day 7 after antibody transfer to potentate the effect of the antibody transfer and to synchronize the onset of arthritis. This model circumvents B- and T-cells responses and provides directly joint specific antibodies able of binding to collagen. This in turn triggers the innate immune responses e.g. chemotaxis of pr-inflammatory cells into the joint and subsequently cartilage degradation in susceptible mice strains. This model has a relatively rapid disease course (30 to 37 days from onset to remission). It was found that the loss of PG during arthritis course was significantly higher in KO mice compared with their WT littermates (FIG. 8a). This was confirmed by measurements of COMP serum levels, which increased during the disease course and were higher in alpha 10 integrin mice compare to WT (FIG. 8b). Again, KO and WT showed similar disease courses (FIG. 6b) peaking at day 12-13.

### Conclusion

All together, the results lead to the conclusion that, alpha10beta1 integrin plays a role in ECM integrity. This role is put to the test in a more obvious way after inflammatory stress when mice ECM had a lower aptitude to recover from PG loss. The absence of alpha10 integrin might unbalance the synthesis-destruction equilibrium, i.e. the ECM matrix turnover, carried out by the chondrocytes. One can hypothesize that the lack of alpha10 beta1 integrin signalling in RA, with recurring disease relapses, will lead to severe cartilage damage.

### Animals

For all the arthritis animal experiments, Itga10 KO mice, described in WO03/101497 incorporated herein by reference, on a C57/bl6 background, (Cartela A B, Lund, Sweden) were backcrossed to B10Q mice for 4 generations. H2q homozygous mice were intercrossed to breed KO mice and littermate controls. The breeding was conducted at the medical inflammation research animal house at Lund University (MIR).

All the animals were kept in a conventional animal barrier facility in polystyrene cages covered with wood shaving. The mice were kept in a 12 h light-dark cycles conditions and an access to chow and water ad libidum. All experiments were conducted respecting the applied ethical law for animal welfare.

Sixteen to eighteen-week old mice were used in all the experiments. Mice were age and sex matched according to the type of experiments.

### Experimental Arthritis Models

### CIA (Collagen Type II Induced Arthritis)

Sixteen to eighteen-week old male mice were immunized with rat type II collagen (CII). CII was diluted with 0.1M acetic acid to a concentration of 2 mg/ml and was emulsified in an equal volume of complete Freund's adjuvant to a final concentration of 1 mg/ml. The mice were immunized intradermally at the base of the tail with 100 μl emulsion (100 μg collagen). The animals were boosted intradermally at the base of the tail with an injection of 50 μg collagen type II diluted at a concentration of 2 mg/ml with 0.1 mol/l acetic acid and was then emulsified in an equal volume of incomplete Freund's adjuvant at day 35.

### AIA (Antigen Induced Arthritis)

Methylated bovine serum albumin (mBSA) (Fluka, Germany) was diluted in a physiological saline solution (0.09% NaCa), and emulsified in an equal volume of complete Freund's adjuvant or incomplete Freund's adjuvant (Difco laboratories, Detroit, USA) to a final concentration of 100 μg in 100 μl. The solutions were injected intradermally at the base of the tail. Mice were additionally injected intraperitoneally with pertussis toxin (Bordella Pertussis, Sigma-Aldrich Chemie, Germany) at 500 ng diluted in 50 μl PBS.

At 14 days after i.d injection the mice were challenged by intra-articular injection of 50 μg mBSA in 5 μl saline into the right knee joint cavity, while the left knee joint was injected with saline alone as a negative control.

### CAIA (Collagen Type II Antibodies Induced Arthritis)

### 4-Antibodies-Transfer CALM

Sixteen to eighteen-week old male mice were injected with a cocktail of antibodies against type II collagen. The combination of anti-type II collagen antibodies used were used (CIIC1, M2139, CIIC2 and UL1). The monoclonal antibodies were mixed at equal concentrations and injected intraveniously as a single dose, resulting in a dose of 1 mg per antibody per mouse. At day 7 the mice were given an intraperitoneally injection of lipopolysaccharide (LPS; E. coli, Sigma) at a concentration of 25 ug/mouse.

### UL1-Antibody-Transfer CAIA

Sixteen to eighteen-week old male mice were injected with a single antibody against type II collagen as described earlier. The antibody UL1 is directed against the U1 epitope of CII. The monoclonal antibody was diluted in sterile PBS at a concentration of 4.5 mg/ml. For the time study mice were sacrificed either at day 0 (no antibody transfer), day 1 (24 h after transfer), day 3 (72 h after transfer) or at day 21 after antibody transfer.

### Assessment of Disease Onset and Development

In CAIA and CIA, mice were clinically scored for arthritis in peripheral joints. The extended scoring system has been described earlier. In short, mice were considered arthritic when significant changes in redness and/or swelling were noted in digits or part of the paws (front and back ones). Clinical severity of arthritis was graded on a scale of 0-15 for each paw (Holmdahl, 1998, pp 215-238). Arthritis score (mean±standard deviation) was expressed as cumulative value for all paws, with a maximum of 60.

In the AIA model both knee joints were measured before and every day after arthritis induction using an Oditest vernier calliper (Kroeplin Längenmesstechnik, Schlüchtern, Germany). Joint swelling was expressed as the difference in diameter (mm) between the right (arthritic) and left (control) knee joint.

### Determination of Anti-Collagen Antibodies

Serum antibodies raised against rat type II collagen were examined using ELISA. Titres of total IgG was measured. Briefly, plates were coated with 10 μl/ml rat collagen type II (50 μl/well). Nonspecific binding sites were blocked with 1% BSA (Sigma Chemicals). Sera were diluted 1:100 times, and added to the plate followed by incubation with isotype-specific goat anti-mouse alkalin phosphatase (Jackson, USA) and phosphatase substrate (Sigma-Aldrich, USA). Plates were read at 405 nm.

### Determination of Seric COMP

Level of serum COMP from the CIA and CAIA mice were determined. Sera were collected either at day 0, 14 and 65 (end point) in the CIA experiment or at day 0, 14 and 37 (end point) in the CAIA experiment. Briefly, plates were coated with 0.2 μg/ml purified rat COMP (50 μl/well). Nonspecific binding sites were blocked with 1% BSA (Sigma Chemicals). Sera were diluted 1:100 times. Sera dilutions were added followed by incubation with a polyclonal rabbit anti-COMP antibody. A swine anti-rabbit alkalin phosphatase labelled antibody (Dako, Denmark) and substrate (Sigma-Aldrich, USA) was used for detection. Plates were read at 405 nm. Concentrations were expressed as means±standard deviation.

### Histology

### CIA

Ankles were removed and fixed for 24 hours in 4% formaldehyde. After decalcification for 3 weeks at 4° C. in 4% formic acid, the specimens were processed for paraffin embedding. Tissue sections (7 μm thick) were stained with Safranin O and haematoxylin. Histo-pathological changes were scored on the ankle at the tibia-talus junctions.

### AIA

For histology knee joints were removed and fixed for 24 hours in 4% formaldehyde. After decalcification for 3 weeks at 4° C. in 4% formic acid, the specimens were processed for paraffin embedding. Tissue sections (7 μm thick) were stained with Safranin O and haematoxylin. Histo-pathological changes in the knee joints were scored on comparable sections of the patella, patella-femur, femur-tibia and tibia region.

### CAIA

Ankles were removed and fixed for 24 hours in 4% formaldehyde. After decalcification for 3 weeks at 4° C. in 4% formic acid, the specimens were processed for paraffin embedding. Tissue sections (7 μm thick) were stained with Safranin O and haematoxylin or for higher sensitivity with Toluidine Blue. Histo-pathological changes were scored at the ankle joints and morphological alterations were assessed. Histo-pathological changes were scored on the ankle at the tibia-talus junctions.

### Histological Scoring System

All histological evaluations were performed on coded slides for blinded scoring and changes were scored using the following parameters. Infiltration of cells was scored on a scale from 0 to 3, depending on the amount of inflammatory cells in the synovial tissues. Inflammatory cells in the joint cavity were graded ranking from 0=no cell infiltration and normal synovium with 2-3 layer of cells; 1=mild infiltration of 5-10 layers of cells; 2=moderate infiltration of 10-15 layer of cells; 3=severe infiltration of cell over 15 layers of cell. (Van den Berg, 2003).

Cartilage proteoglycans depletion was determined using safranin-O staining. The loss of proteoglycans was scored on a scale from 0 to 3 according to loss of staining intensity: 0=fully stained cartilage; 1=mild reduction of safranin O staining; 2=moderate reduction of safranin O staining; 3=complete loss of safranin O staining.

For the analysis of articular cartilage erosion, cartilage destruction was assessed separately and scored on a scale from 0 to 3: 0=no abnormalities on the cartilage surface; 1=mild irregularities; 2, moderate cartilage destruction; 3=complete cartilage destruction.

The presence of osteophyte was rated as follow: 0=no osteophyte formation; 1=less then 2 osteophytes of small size; 2=several osteophytes number (more than 3) or presence of large osteophytes.

Histo-pathological changes were scored on the ankle at the tibia-talus, talus-calcaneus, calcaneus-first cuboid junctions (CIA and CAIA) and in the knee at patella, patella-femur, femur-tibia and tibia region (AIA).

For each joints, the entire surface of the cartilage was scored. Each joint histological status was assessed according to the histological scoring system. The average of the scores of the entire joint for each single paw ranked between 0 and 3 for all the criterion except for osteophytes presence which scores ranged from 0 to 2.

Average of score for all KO mice and average of score for all WT mice were determined and converted in percent of maximal possible score (3 points for all except for osteophyte presence which max score was 2 points).

### Statistical Analysis

Differences between experimental groups were tested using the Mann-Whitney U test.

### Results

### Absence of Integrin Alpha10 Affects PG Depletion and Cartilage Destruction

To investigate the ability of the alpha 10 integrin knock out (Itga10 KO) mice to develop arthritis CIA was induced by injection of type II collagen subcutaneously followed by a boost immunization at day 35. The mice were scored for arthritis during 64 days. To assess the capacity of the mice to respond to type II collagen, serum level of anti-collagen II antibodies were determined for two chosen time points. Serum level at day 0 was compared with serum level at day 14 in the same mice after immunization. The Itga10 KO on B10Q background (B10.Q_alpha10) and wild type littermates did not show difference in antibody responses to type II collagen. All groups produced similar levels of anti-collagen II antibodies (FIG. 1).

No significant difference in severity of arthritis was observed between the C57Bl10.Q wild type (WI) mice, Itga10 KO or mice heterozygous for the knocked out Itga10 gene (HET) (FIG. 2.a). The incidences in all three groups were also similar. At the end point of the experiment, 5 out of 11 KO (i.e. 45.5%), 3 out of 9 WT (i.e. 33.3%) and 15 out of 30 HET mice were sick (i.e. 50%) (FIG. 2.b).

To evaluate the influence of integrin alpha10beta1 on tissues damage of the arthritis, hind paws from al KO and WT mice were collected at the end of experiment for histological analysis. Sections were stained with Safranin O and scored blinded. To rate the degree of tissue damage four distinct criteria were used: inflammation level (infiltration of cells into the articular joints), cartilage erosion and/or destruction, proteoglycan depletion and formation of osteophytes (see material and methods). that the absence of integrin alpha10beta1 10 did not play a role in how effectively cells were invading and homing in the joint space, since mutant mice showed the same proliferating of synoviocytes as WT littermate (data not shown). No differences in osteophyte formation were detectable between the Itga10 KO and WT littermates (data not shown). However, proteoglycan depletion as well as cartilage degradation seems to be higher in mutated mice (FIG. 3.a). There was a strong tendency for proteoglycan depletion differences between KO (51%) and WT (28%). Cartilage degradation in KO (38%) compare to the WT (19%) was significant.

Both Itga10 KO and WT mice had increased COMP serum levels already at day 14 after collagen type II immunization. The COMP-levels were significantly higher in KO in sera collected at experiment endpoint (P=0.050) (FIG. 3.b).

### Absence of Integrin Alpha10 Chain Increases Proteoglycan Depletion in Antigen-Induced Arthritis (AIA)

In order to further investigate the role of integrin alpha10beta1 in arthritis development and progression, AIA was induced in Itga10 KO mice. WT littermates were used as controls. In AIA, arthritis is rapidly developing in the knee joint challenged by intra articular injection of methylated BSA (mBSA) while the unchallenged knee stays unaffected. The mice were sensibilities with mBSA 14 days prior to the intra articular injection. The knee joint injected with PBS is used as intend control.

Firstly, the induction protocol was optimized. Immunization with mBSA emulsified in either complete Freund adjuvant (CFA), complete Freund adjuvant plus Pertussis toxin (CFA+Ptx) or incomplete Freund adjuvant (IFA). The different adjuvants used showed only a slight variation in swelling. The mildest swelling result was obtained when mice were injected mBSA together with IFA. Mice injected with mBSA with CFA or mBSA with CFA plus Ptx gave similar arthritis scores with a slightly more potent swelling with CFA alone as adjuvant (data not shown). For the further experiments, disease was induced by mBSA in CFA.

There was no difference in joint swelling after AIA induction between the Itga10 KO, WT and HET mice (FIG. 4). All mice developed joint swelling with the incidence of 100%.

At termination of the experiment, knees were collected in order to investigate histological variation in KO compare to WT mice. Knee sections were stained with Safranin O and scored according to the same rating system as in CIA experiment taking into account the following criteria: cell infiltration, proteoglycan depletion, cartilage degradation and osteophyte formation.

Cell invasion into the interstitial knee joint was not altered by the absence of alpha 10 integrin. Observation of joint cartilage showed that alpha 10 integrin mutant mice were significantly more affected and showed more reduction of proteoglycan compare to WT controls (FIG. 5). No cartilage destruction and no osteophytes formation were detected.

### Absence of Integrin Alpha 10 Integrin did not Affect the Severity of 4 Antibodies—Collagen Antibody Induced Arthritis (4Ab-CAIA)

Induction of CAIA in the Itga10 KO mice and its WT littermates showed that the cocktail of four antibodies (M2139, CIIC1, UL-1 and CIIC2) recognizing respectively J1, C1, U1 and D3 epitopes on type II collagen, did not promote different inflammatory responses in Itga10 KO mice compare with WT littermate. The severity and duration of arthritis was identical for both groups during the whole disease course (FIG. 6a). However, the Itga10 KO mice had a tendency of higher incidence before and right after the LPS injection. The onset of arthritis was slightly earlier in the integrin Itga10 KO mice compared to the WT littermates, although not significant. The number of Itga10 KO arthritic mice increase in score until day 12 and 13 where all the mice were sick. For the WT littermate mice at the same time point 14 out of 15 were sick (FIG. 6b).

For both KO and WT groups, both sexes were included in the experiment. When analyzing the results separating males and females, the scoring showed no difference in WT between the sexes. Both males and females got equally sick and for the same duration of time. The incidence showed that WT males were slightly more prone to get sick than WT females at the same time point in the disease course. Mutant mice on the other hand showed a difference in severity between both sexes. Males showed tendency for higher scores during the whole disease duration which was not significant. The Itga10-KO males have also a higher incidence compared to Itga10-KO females before and after LPS injection (data not shown).

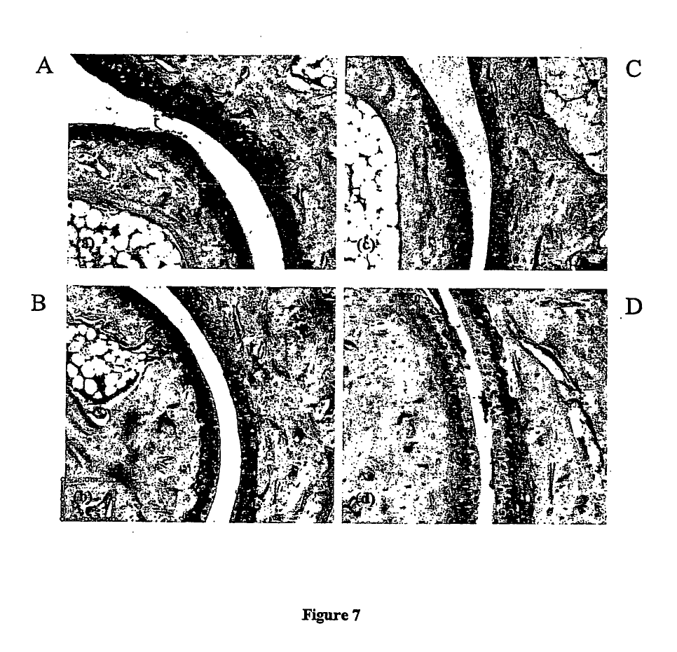

Histology sections of 4Ab-CAIA (CIIC1, M2139, CIIC2 and UL1) showed depletion of PG in both KO and WT littermates (FIG. 7). The stainings are from the experiment endpoint at day 37. As seen in CIA and AIA, PG depletion was significantly increased in KO mice. In addition, there was a tendency for increased cartilage destruction in KO mice compare with the WT littermates. No osteophytes were observed in these sections (FIG. 8a). Serum analysis showed that COMP levels were similar at day 0 and increased at day 14 and up to day 37. Results showed that the COMP concentration were higher in KO than in WT in a significant manner at day 14 and day 37 (FIG. 8b).

### Absence of Integrin Alpha10 Chain on UL1 Antibody Collagen Antibody Induced Arthritis (UL1 Ab-CAIA)

Antibody transfer with UL-1 anti collagen II antibody (directed against U1 epitope) has been shown to be a model were PG are depleted as early as 24 h after transfer of the single monoclonal UL-1. In this experiment time dependant PG depletion in both Itga10 KO and WT were observed. Results showed that both KO mice and WT showed PG depletion at day 3 after antibody transfer with a tendency of higher loss in alpha 10 integrin deficient mice. At day 7 WT acquired again a normal PG content in the cartilage which looked similar to the staining at day0. In the Itga10 KO mice however, there were indication of reduced rate of PQ synthesis since normal levels of PG were not restored until day 21.

### Discussion

It is show that absence of the alpha 10 integrin, which is mainly expressed by chondrocytes in cartilage, did not seem to affect the onset or the course of induced arthritis in any of the animal models studied here. Despite the deletion of alpha 10 integrin in heterozygous or homozygous manner, the mice developed similar disease as their wild type littermates. Arthritis severity revealed no differences and similar paw scoring were recorded for both alpha 10 integrin KO and WT mice. Both the CIA and the 4Ab-CAIA experiment seemed to show a slightly higher disease incidence for the KO mice however this difference was not significant. Moreover, arthritis onset in both experiments was similar for alpha 10 integrin KO and WT.

In arthritis, inflammation and swelling of the joint are due to abnormal proliferation of synoviocytes (Fibroblast like synoviocytes, FLS and Macrophages like synoviocytes, MLS) and a massive invasion of inflammatory cells. Histological scoring for inflammation in ALA, CIA and CAIA experiments showed that joint invading cells in the interstitial joint space were similar in the WT and alpha 10 integrin KO mice. These histological observations also reinforced our finding that the arthritis manifestations in the mice were not affected by the absence of alpha 10 integrin. Despite the apparent nonexistence of alpha10 integrin implication in inflammation process, it is important to mention that the observation of histological section in these arthritis models were made at experiment endpoint and that the implication of alpha 10 integrin in inflammation might happens in a more discreet way or in an other time window.

Further histological analysis of disease-affected joints from alpha 10 integrin deficient mice showed increased proteoglycan depletion at the cartilage surface, judged by reduced Safranin O staining, which was more severe than the PG depletion observed in the WT mice. This enhanced reduction in PG content in alpha 10 integrin KO mice compared to WT mice was conclusive from all the performed RA experiments. It is not known if alpha 10 integrin can interact directly with proteoglycans. Nevertheless, collagen fibrils have been described to interact with keratan sulfate rich regions of several aggrecan (large chondroitin sulfate proteoglycan) associated in PG aggregates and that these fibrils might serve as backbone structure for some aggrecan complexes within cartilage reinforcing the complex assembled aggrecan molecules network. The absence of alpha 10 integrin might partly result in disorganization of collagens and subsequent PG aggregate disturbance in the articular cartilage rending it more prone to enzymatic degradation as it happens in arthritis.

Proteins constituting the matrix are assembled in an intricate mesh and therefore the reduction or total absence of one component of the matrix can in turn disturb the composition of several other components of the matrix. Specific staining of alpha 10 integrin mutant mice with antibodies against collagen type II, matrilin-1, matrilin-3 and aggrecan have been performed earlier but this didn't reveal any differences in expression intensity or distribution of these matrix molecules between WT and alpha 10 integrin KO mice. However, these staining were all done in newborn mice at an age where the cartilage is still under development. In our study, the loss of proteoglycan could lead to several subsequent events. Proteoglycan are found in the cartilage in aggregate composed of aggrecan, link protein, and hyaluronan. These aggregate allow the tissue to have gel like property that provides resistance to compression in joints and give the cartilage a structure critical for growth plate formation Aggrecan is one of the major structural macromolecules in cartilage and binds hyaluronan and link protein as well as CII containing fibrils via matrilisin-1. Mouse cartilage matrix deficiency (cmd) resulting from a functional null mutation of the aggrecan gene resulted in an important chondrodisplasia characterized by perinatal lethal dwarfism. Analysis of these mice revealed that the proteoglycan aggregate plays an important role in cartilage development and maintenance of cartilage tissue and that the cmd mice showed a disturbed collagen-fibril expression pattern suggesting a role of aggrecan in collagen fibril formation. The observations made in the aggrecan deficient mice witnessed among other things an evident implication of the growth plate disturbance reminding the observation made earlier in alpha 10 integrin KO. Maybe the absence of alpha 10 integrin has disturbed the PG composition already from embryonic stage and all the way through adult stage in a mild fashion. Observations on new born mice showed a mild growth plate dysfunction and a reduced long bone size. The disturbed bone growth is maintained through adult stage and is visible in a significant manner in 12 weeks old and 1 year old mice.

Alpha 10 integrin KO (n=3) and WT littermate (n7-3) were transferred with UL-1 antibody, an antibody directed against type II collagen U1 epitope. The U1 epitope have been reported to overlap with integrins preferential binding epitope on type II collagen. The depletion of PG in Itga10 KO and its WT littermates were equal (day 3 after injection). In the WT mice the process of restoring the cartilage had begun and the level of PG was almost normal at day 7 after antibody injection. In the Itga10 KO mice however, the restoration of the levels of PG in the cartilage were slower. At day 7 no improvement of the PG level were observed while at day 21 cartilage seem to come back to level of PG prior to UL-1 antibody. The slower return to original level of PG contain could be due principally to an impaired signalling through alpha 10 integrin but also maybe to subsequent impairment of other integrins on chondrocytes surface since down regulation (by internalization) of integrins on cell surfaces could have happened due to the mild abnormal matrix composition of the alpha 10 integrin. Studies have shown that, reduction or total impairment of specific ligands could lead to down regulation of integrins cell surface expression that they normally bind too. Moreover, at the light of these results, it seems that alpha 10 integrin might play a role in PG de novo synthesis. This can also be the case in CIA and AIA experiments which showed a more important loss of PG. It is possible that these results reflected the lower capacity of alpha 10 integrin to recover from cartilage damage by PG de novo synthesis.

In arthritic conditions, the presence of IL-1 has been shown not only to inhibit aggrecan and CII synthesis but also to increase MMP production. The subsequent cartilage degradation leads to production of small fragments of extra-cellular matrix that have been shown to have the ability to up-regulate cytokine expression, down-regulate or suppress matrix synthesis and increase MMP production accentuating the initial effect of IL-1 on cartilage. For instance, fibronectin fragment have been shown to modulate expression of the collagenase MMP-13 and to induce aggrecan loss after degradation by aggrecanase and not MMP's (according to fragments resulting from the degradation) in cartilage explants. It has been shown earlier that proteinases produced in physiological situations by chondrocytes, such as MMP13, having an important role in growth plate remodelling and endochondral ossification and that these proteinase synthesis are several fold increased in arthritic tissue. In this approach, the absence of alpha 10 integrin in RA models is maybe leading to up regulation of integrins able to bind to small fragments generated from ECM proteins degradation that take place in RA, leading to different cell signalling hence excessive and rapid MMP synthesis.

The alpha 10 integrin KO might have difficulty to compensate quickly enough the loss of ECM components happening in the stressed arthritis cartilage joint leading ultimately to a better possibility for MMP's and other matrix degrading proteinases to access the matrix and to destroy it more efficiently.

COMP, a non-collagenous protein, is measured in the serum and is the result of the degradation of cartilage. It has been described to be in high level in patients in early phase of RA development. Enhanced loss of cartilage proteoglycans in alpha 10 integrin deficient mice was confirmed by analysis of COMP in the serum. Both CIA and 4Ab-CAIA experiments showed that COMP increased during the course of induced arthritis. The levels measured in CIA experiment were higher than in CAIA and are the representation of a more aggressive process happening in CIA where several cellular and humoral routes of inflammation are solicited in concert. COMP serum presence in latest stage of CIA and 4Ab-CAIA reflects progressive and continuous cartilage degradation and that even after visible complete inflammation disappearance (4Ab-CAIA endpoint day 37). The COMP levels were higher at all time point in alpha 10 integrin KO reaching significance for 4Ab-CAIA already at day 14 after disease onset and staying significant at experiment endpoint. In the CIA experiment, significant difference between alpha 10 KO and WT littermate was reached at day 65. COMP level normally decreases in patients after the acute phase.

Finally, osteophytes are osteo-cartilaginous tissues which formation is started by chondrogenenic differentiation of periosteum or synovium derived mesenchymal cells. This process is often very apparent in models showing important bone destruction such as osteoarthritis (OA), in type IX collagen mutant mice bearing histological characteristics similar to those of OA or in model induce with injection of TGF-beta1. In the present study, osteophyte formation was observed only in the CIA model. In this model, the size and average numbers of ostephytes did not differ in the Itga10 KO compared to its WT littermates, despite the cartilage destruction accompanied by important bone destruction in our CIA experiments. It seems that the absence of alpha 10 integrin do not disturb the formation of new bone formation triggered by cartilage and bone destruction.

### Example

### Production of Exemplary Antibodies Binding Alpha10

Identification of n-CoDeR®-Derived Human Antibodies Specific for α11β1integrin

The fully human antibody library n-CoDeR® (see Soderlind et al., 2000, Nat Biotechnol 18:852-6 and WO 98/32845, which are incorporated herein by reference) was screened for antibody fragments (scFv) with specificity for the α10β1 integrin. A series of combined positive and subtractive panning methodologies was devised in order to maximise retrieval of highly target integrin specific antibodies. Following conversion to scFv format, antibody fragments were expressed in E. coli and were positively or negatively screened for binding to Hek-293 cells expressing α10β1 integrin, using FMAT technology (FMAT scatter plot). Thus, hundreds of genotype unique antibody fragments specific for al Op integrin were identified.

### Antibody Panning Procedure

A phage stock of n-CoDeR® scFv library in a buffer of 3% BSA, 0.02% sodium azide, 0.1% NP40, 10 mM MgCl₂ and 0.01 mM CaCl₂ in PBS was pre-selected over night at 4° C. using BSA coated in an immunotube and tosyl activated Dynabeads M-280 (Dynal Cat # 142.04) coupled with rabbit-anti-alfa10 integrin polyclonal antibodies according to Dynals' instructions. The first panning was performed over night at 4° C. on lysate from 15×10⁶ HEK293 cells expressing α₁₀β₁ loaded on magnetic beads coupled with rabbit-anti-alfa10 polyclonal antibody (as above). Beads were washed with 9×1 ml buffer and bound phage were eluted with trypsin and amplified as described elsewhere (Hallborn & Carlsson, 2002, Biotechniques December; Suppl:30-37).

Amplified phage from the 2^{nd} panning, buffered as above, were pre-selected on polyclonal coupled Dynabeads as in 1^{st} panning followed by a selection analogous to the first panning with lysate from 10×10⁶ HEK293 α₁₀β₁, cells loaded on the Dynabeads with rabbit-anti-alfa11 polyclonal antibody. After washing (9×1 ml buffer) bound phage were eluted with trypsin and amplified.

Amplified phage from the 2^{nd} panning, diluted in DMEM cell medium containing 10% FCS, 10 mM MgCl₂ and 10 μM CaCl₂, were pre-selected over night at 4° C. against 45×10⁶ C2C12 cells expressing α₁₁β₁ and 13×10⁶ HEK cells expressing α₁₁β₁. Phages were then incubated for 4 h at 4° C. with α₁₀β₁ displayed on recombinant C2C12 cells (5×10⁶ cells used). After density centrifugation through 40% Ficoll (containing 2% FCS, 10 mM MgCl₂ and 10 μM CaCl₂) to remove unbound phages, remaining binders were eluted with both 76 mM citric acid pH 2.5 and 200 mM triethanolamine. The pool of eluted phages was amplified in Escherichia coli HB101F′ for conversion to scFv format.

Conversion to scFv Format

Conversion to c-myc/6×his scFv format (EagI conversion) Protein III is the functional link between the scFv and the phage particle and removal of gene III results in production of soluble scFv. Phagemid DNA from the n-CoDeR Lib2000 selection is digested with EagI to remove gene III and bring the 6×his tag next to the scFv fragment and c-myc tag. The plasmid is ligated and a “killer-cut” with EcoRI, which has a restriction site within gene mL, is done to eliminate re-ligated phagemids. The plasmid DNA is transformed into chemically competent Escherichia coli TOP10.

### Example

### In Vitro Data on Human Chondrocytes

Human integrin alpha10 specific recombinant IgG4-antibodies prepared from the n-CoDeR Lib2000 selection above were tested for ability to regulate chondrocyte extracellular matrix synthesis and degradation. The strategy is to use either isolated human chondrocytes cultured in high-density monolayer or human cartilage explants from donors with normal or mild OA cartilage. Explant data is found in Example 3.) below.

### Preparation of Human Chondrocytes

Human primary chondrocytes are isolated from femoral condyle. The cartilage is washed 3× in PBS−+1:100 PEST+1:250 fungizone+10% FCS. The cartilage is dissected into ˜1 mm³ pieces. The tissue pieces are Pronase E treated in 700 IU/ml (=10 mg/ml) dissolved in DMEM-F12+1:100 PEST+1:250 fungizone+5% FCS, pre-warmed solution to 37° C. before using. After 30 minutes incubation at 37° C., the supernatant is removed and discarded. The pieces are washed three times in DMEM:F12+1:100 PEST+1:250 fungizone+10% FCS to remove all pronase. Collagenase (Sigma C9891—175 U/ml for older tissue; ˜100 U/ml for young tissue) dissolved in DM-F12+1:100 PEST+1:250 fungizone+5% FCS, sterile filtered and prewarmed solution is used to further treat the pieces. The chondrocytes are then plated out onto plastic after resuspension and final wash in DMEM-F12+10% FCS+1:100 PEST+1:2000 ascorbic acid and put in flask at 37° C. The cells were plated at high density (8-10×10⁶ cells/12 ml) and low density 5-6×10⁶ cells/12 ml The cells were grown for ˜5 days before freezing/experiment.

### Monolayer Cultures

In the high-density monolayer culture system human chondrocytes are isolated as above and expanded in monolayer for 4-5 days before seeding in 96-well culture plates (>180000 cells/well) and stimulation with antibodies or control substances were used. This is a short-term experiment (48 hours) with a single treatment with antibodies (5 μg/ml). We analyse antibodies for effect on newly synthesised proteoglycans (i.e. 35-S incorporation for 4 hours). Chondrocytes from two human donors (61 and 63 y.o.) have been analyzed. Taken together these data show that one antibody (IgG4-A05) up-regulate proteoglycan synthesis (p<0.001) compared to the CT17 control IgG4. Statistical analyses: one-way analysis of variance with Dunnett's Multiple Comparison Test., for stat significance in individual experiments—see below).

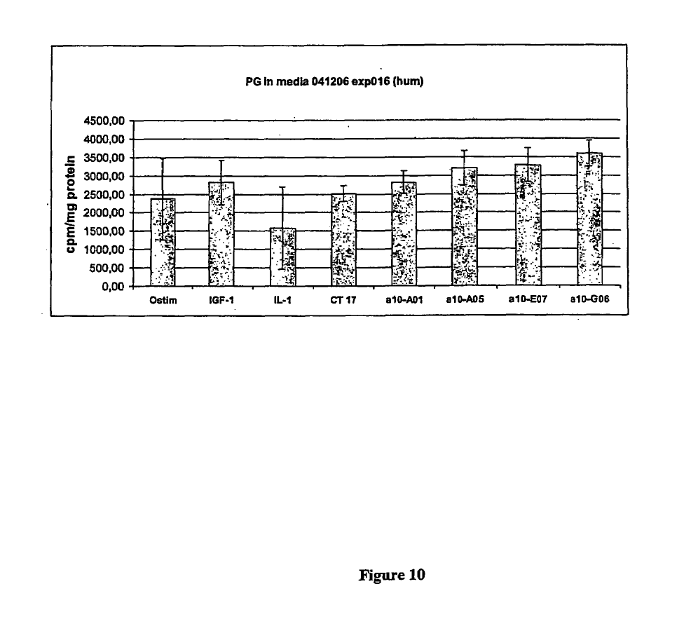

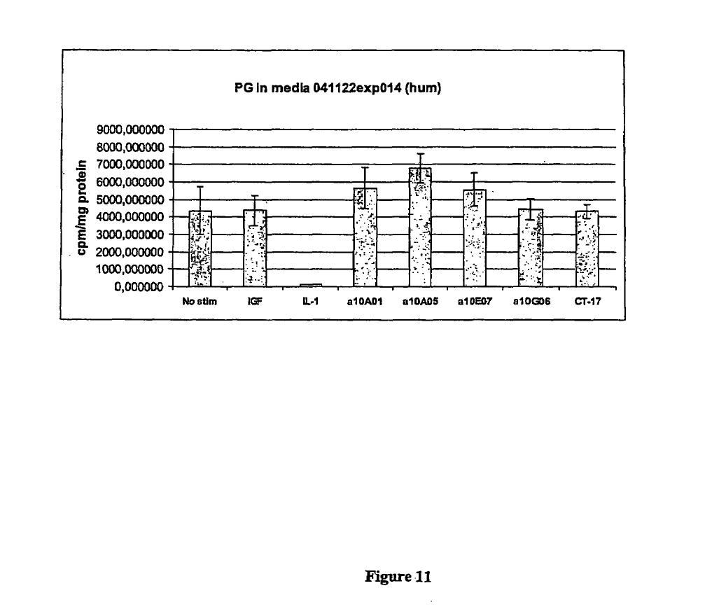

Results from two independent experiments of human chondrocytes are shown. FIGS. 10 and 11 shows exp014 & exp016. Production of newly synthesized proteoglycan in response to alpha10 binders. IGF-1 (400 ng/ml) was used as a positive controls and IL-1β (10 ng/ml) was used as a control for down-regulation of proteoglycan synthesis. Proteoglycan synthesis (4 h 35-S incorporation) was analysed after a single treatment with IgG4 (5 μg/ml) or svFv (10 μg/ml, except scFv-E07 that was used at 5 μg/ml) for 48 h. Mean values with s.e.m are plotted. Statistical analyses: t-test.

Experiment—In Situ Data from Explants

Human explants, i.e. cartilage cut into 1-2 mm³ pieces and kept in culture for up to four weeks. The chondrocytes are situated in their normal context surrounded by the natural cartilage matrix components. The chondrocytes keep their state of differentiation and the expression of e.g. integrins is unlikely to change as it changes along with the dedifferentiation process during monolayer culture. Explant cultures also makes it possible to study cartilage and chondrocytes from patients with different degree of osteoarthritis.

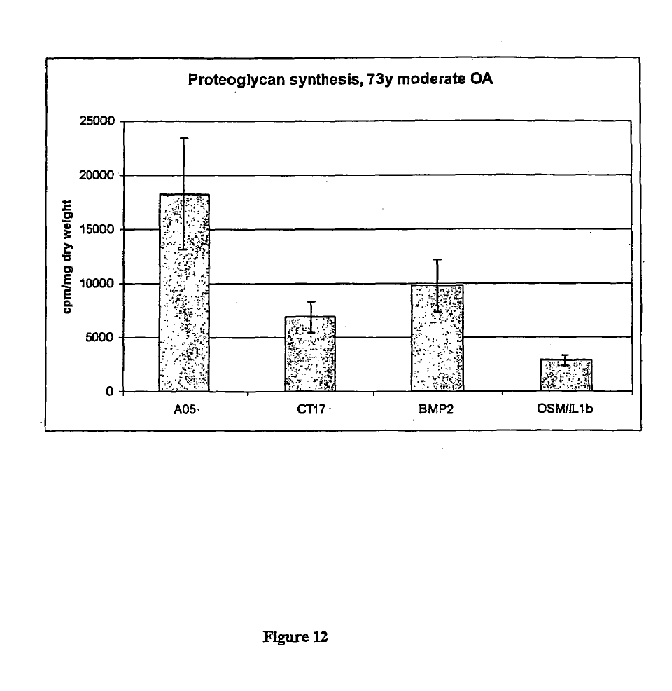

In the present study specimens with no to moderate osteoarthritis have solely been used. The explants were treated with A05 for 48 hours and pulsed with 35-S during the final four hours. IgG4-A05 significantly up-regulates proteoglycan synthesis compared to the IgG4 control (p<0.05) (FIG. 12 below).

In conclusion, the integrin alpha10 binder A05 have so far had statistically significant effects on matrix synthesis after conversion to IgG4 both on monolayer cultured chondrocytes (FIG. 10-11) and on human cartilage explants (FIG. 12).

1.-35. (canceled)

36. A binding entity binding specifically to integrin subunit alpha 10, or a heterodimer thereof, for treatment, detection, diagnosis or prognosis of a condition wherein cartilage proteoglycan turnover is affected.

37. The binding entity of claim 36, wherein the binding entity is an antibody, a peptide, or a collagen moiety.

38. A pharmaceutical composition comprising the binding entity of claim 36 and at least one pharmaceutically acceptable excipient, diluent or carrier.

39. A method for modulating cartilage proteoglycan turnover, comprising the steps of
a) providing cartilage proteoglycan comprising cartilage cells, and
b) targeting integrin subunit alpha 10, or a heterodimer thereof, on said cells with the binding entity of claim 36, for sufficient time to modulate cartilage proteoglycan turnover.

40. The method of claim 39, wherein the cartilage proteoglycan turnover is modulated to inhibit, prevent or slow down, or reduce degradation of proteoglycan.

41. The method of claim 39, wherein the cartilage proteoglycan turnover is modulated to increase de novo synthesis of proteoglycan.

42. A method for treating a condition affecting cartilage proteoglycan turnover, comprising administering to a patient having such a condition an effective amount of the pharmaceutical composition of claim 38.

43. The method of claim 42, wherein the condition is osteoarthritis or rheumatoid arthritis.

44. A method for detecting a condition affecting cartilage proteoglycan turnover in an individual, comprising the steps of
a) providing cartilage proteoglycan comprising cartilage cells from an individual,
b) targeting integrin subunit alpha 10, or a heterodimer thereof, on said cells with the binding entity of claim 36,
c) detecting said binding entity, and
d) scoring cartilage proteoglycan turnover compared to a reference sample, thereby detecting said condition affecting cartilage proteoglycan turnover.

45. The method of claim 44, wherein the condition is osteoarthritis or rheumatoid arthritis.

46. A method for diagnosing or prognosing a condition affecting cartilage proteoglycan turnover in an individual, comprising the steps of
a) providing cartilage proteoglycan comprising cartilage cells from an individual,
b) targeting integrin subunit alpha 10, or a heterodimer thereof, on said cells with the binding entity of claim 36,
c) detecting said binding entity,
d) scoring cartilage proteoglycan turnover compared to a reference sample, and
e) relating said scoring in d) to a diagnosis or a prognosis of a condition affecting proteoglycan turnover.

47. The method of claim 46, wherein the condition is osteoarthritis or rheumatoid arthritis.
